(19) United States
(12) Patent Application Publication
(10) Pub. No.: US 2007/0295251 A1
(43) Pub. Date: Dec. 27, 2007

(54) EPSP SYNTHASES: COMPOSITIONS AND METHODS OF USE

(75) Inventor: Volker Heinrichs, Raleigh, NC (US)

Correspondence Address: ALSTON & BIRD LLP, BANK OF AMERICA PLAZA, 101 SOUTH TRYON STREET, SUITE 4000, CHARLOTTE, NC, 28280-4000, US

(73) Assignee: Athenix Corporation, Research Triangle Park, NC (US)

(21) Appl. No.: 11/762,526

(22) Filed: Jun. 13, 2007

Related U.S. Application Data
(60) Provisional application No. 60/878,259, filed on Jan. 3, 2007.
(60) Provisional application No. 60/813,061, filed on Jun. 13, 2006.

(51) Int. Cl. A01G 23/02; A01H 1/00; A01H 5/00; C07H 21/02; C07H 21/04; C12N 5/04

(52) U.S. Cl. 111/100; 435/413; 435/418; 536/23.1; 536/23.6; 800/278; 800/314; 800/315; 800/320; 800/320.1; 800/320.2; 800/320.3; 800/322

(57) ABSTRACT

Compositions and methods for conferring tolerance to glyphosate in bacteria, plants, plant cells, tissues and seeds are provided. Compositions include novel EPSP synthase enzymes and nucleic acid molecules encoding such enzymes, vectors comprising those nucleic acid molecules, and host cells comprising the vectors.

### CROSS REFERENCE TO RELATED APPLICATION

This application claims the benefit of U.S. Provisional Application Ser. No. 60/878,259, filed Jan. 3, 2007, and U.S. Provisional Application Ser. No. 60/813,061, filed Jun. 13, 2006, the contents of which are herein incorporated by reference in their entirety.

### REFERENCE TO SEQUENCE LISTING SUBMITTED ELECTRONICALLY

The official copy of the sequence listing is submitted electronically via EFS-Web as an ASCII formatted sequence listing with a file named “329213_SequenceListing.txt”, created on Jun. 8, 2007, and having a size of 78 kilobytes and is filed concurrently with the specification. The sequence listing contained in this ASCII formatted document is part of the specification and is herein incorporated by reference in its entirety.

### FIELD OF THE INVENTION

This invention relates to plant molecular biology, particularly novel EPSP synthase polypeptides that confer improved resistance or tolerance to the herbicide glyphosate.

### BACKGROUND OF THE INVENTION

N-phosphonomethylglycine, commonly referred to as glyphosate, is an important agronomic chemical. Glyphosate inhibits the enzyme that converts phosphoenolpyruvic acid (PEP) and 3-phosphoshikimic acid (S3P) to 5-enolpyruvyl-3-phosphoshikimic acid. Inhibition of this enzyme (5-enolpyruvylshikimate-3-phosphate synthase; referred to herein as “EPSP synthase”, or “EPSPS”) kills plant cells by shutting down the shikimate pathway, thereby inhibiting aromatic amino acid biosynthesis.

Since glyphosate-class herbicides inhibit aromatic amino acid biosynthesis, they not only kill plant cells, but are also toxic to bacterial cells. Glyphosate inhibits many bacterial EPSP synthases, and thus is toxic to these bacteria. However, certain bacterial EPSP synthases have a high tolerance to glyphosate.

Plant cells resistant to glyphosate toxicity can be produced by transforming plant cells to express glyphosate-resistant bacterial EPSP synthases. Notably, the bacterial gene from Agrobacterium tumefaciens strain CP4 has been used to confer herbicide resistance on plant cells following expression in plants. A mutated EPSP synthase from Salmonella typhimurium strain CT7 confers glyphosate resistance in bacterial cells, and confers glyphosate resistance on plant cells (U.S. Pat. Nos. 4,535,060; 4,769,061; and 5,094,945).

Variants of the wild-type EPSP synthase enzyme have been isolated which are glyphosate-tolerant as a result of alterations in the EPSP synthase amino acid coding sequence (Kishore and Shah (1988) Annu. Rev. Biochem. 57:627-63; Wang et al. (2003) J. Plant Res. 116:455-60; Eschenburg et al. (2002) Planta 216: 129-35). U.S. Pat. No. 6,040,497 reports mutant maize EPSP synthase enzymes having substitutions of threonine to isoleucine at position 102 and proline to serine at position 106 (the “TIPS” mutation. Such alterations confer glyphosate resistance upon the maize enzyme. A mutated EPSP synthase from Salmonella typhimurium strain CT7 confers glyphosate resistance in bacterial cells, and is reported to confer glyphosate resistance upon plant cells (U.S. Pat. Nos. 4,535,060; 4,769,061; and 5,094,945). He et al. ((2001) Biochim et Biophysica Acta 1568:1-6) have developed EPSP synthases with increased glyphosate tolerance by mutagenesis and recombination between the E. coli and Salmonella typhimurium EPSP synthase genes, and suggest that mutations at position 42 (T42M) and position 230 (Q230K) are likely responsible for the observed resistance. Subsequent work (He et al. (2003) Biosci. Biotech. Biochem. 67:1405-1409) shows that the T42M mutation (threonine to methionine) is sufficient to improve tolerance of both the E. coli and Salmonella typhimurium enzymes. Due to the many advantages herbicide resistance plants provide, herbicide resistance genes improved glyphosate resistance activity are desirable.

### SUMMARY OF INVENTION

Compositions and methods for conferring resistance or tolerance to are provided. Compositions include EPSP synthase enzymes that are resistant to glyphosate herbicide, and nucleic acid molecules encoding such enzymes, vectors comprising those nucleic acid molecules, and host cells comprising the vectors. The compositions of the invention include EPSP synthase enzymes other than SEQ ID NO:1 and 46 having the sequence domain X-C-X-E-S-G-L-S-X-R-X-F-X-P-X (SEQ ID NO:44), where X denotes any amino acid. In some embodiments, the EPSP synthase enzymes comprise the sequence domain D-C-X₁-X₂-S-G (SEQ ID NO:76), wherein X₁ denotes glutamine, valine, proline, glutamic acid, isoleucine, methionine, or threonine and X₂ denotes any amino acid. In other embodiments, the EPSP synthase enzymes of the invention comprise the sequence domain X₁-C-X₂-E-G-L-S-X₃-R-X₄-F-X₅-P-X₆ (SEQ ID NO:45) where X₁ denotes D, K, E, S, G, P, R, or N, and X₂ denotes G, Q, V, D, E, I, N, M, A, T, S, or R, and X₃ denotes I, G, S, M, F, or V, X₄ denotes M, A, S, G, Q, L, V, or I, X₅ denotes T, P, L, G, A, V, or I, and X₆ denotes I, L, C, A, F, or M.

Compositions also include nucleic acid molecules encoding herbicide resistance polypeptides, including those encoding polypeptides other than SEQ ID NO: 1 and 46 comprising SEQ ID NO:5-43 and SEQ ID NO:56-65, as well as the polynucleotide sequences of SEQ ID NO:3, 4, 66, 67, 74, and 75 and polynucleotide sequences comprising SEQ ID NO:68-73. The coding sequences can be used in DNA constructs or expression cassettes for transformation and expression in organisms, including microorganisms and plants. Compositions also comprise transformed bacteria, plants, plant cells, tissues, and seeds that are glyphosate resistant by the introduction of the compositions of the invention into the genome of the organism. Where the organism is a plant, the introduction of the sequence allows for glyphosate containing herbicides to be applied to plants to selectively kill glyphosate sensitive weeds or other untransformed plants, but not the transformed organism. The sequences can additionally be used a marker for selection of plant cells growing under glyphosate conditions.

Methods for identifying an EPSP synthase enzyme with glyphosate resistance activity are additionally provided. The methods comprise identifying additional EPSP synthase sequences that are resistant to glyphosate based on the presence of the domain of the invention.

### DESCRIPTION OF FIGURES

FIG. 1 illustrates the combinatorial mutagenesis strategy for the Q-loop region of syngrg1-SB.

FIG. 2 illustrates the design of the permutational mutagenesis library for the Q-loop region of syngrg1-SB. The consensus translation and oligonucleotide design are shown at the bottom of FIG. 2 and in SEQ ID NO:48 (consensus translation) and SEQ ID NO:49 (oligonucleotide design).

FIG. 3 shows the combinatory mutagenesis strategy for Library 3. “N” represents the nucleotide bases A, T, C, or G; “W” represents the nucleotide bases A or T; and, “S” represents the nucleotide bases C or G.

FIG. 4 shows an alignment of the amino acid sequences in the Q-loop core region of the glyphosate resistant clones. The bracket outlines the Q-loop core region. Grey shading designates positions where no alterations are observed. Positions with alterations are shown with no shading. Also included is the wild-type GRG1 amino acid sequence in this region (corresponding to amino acid positions 81 through 104 of SEQ ID NO:2).

FIG. 5 shows an illustration of the crystal structure of GRG1 simulated by layering the GRG1 amino acid sequence upon the E. coli AroA crystal structure (Stallings et al. (1991) Proc Natl Acad Sci USA. 1; 88(11): 5046-50). Panel A shows the full protein relative to the substrates shikimate-3-phosphate and PEP. Panel B shows solely the Q-loop region of GRG1 relative to the substrates shikimate-3-phosphate and PEP.

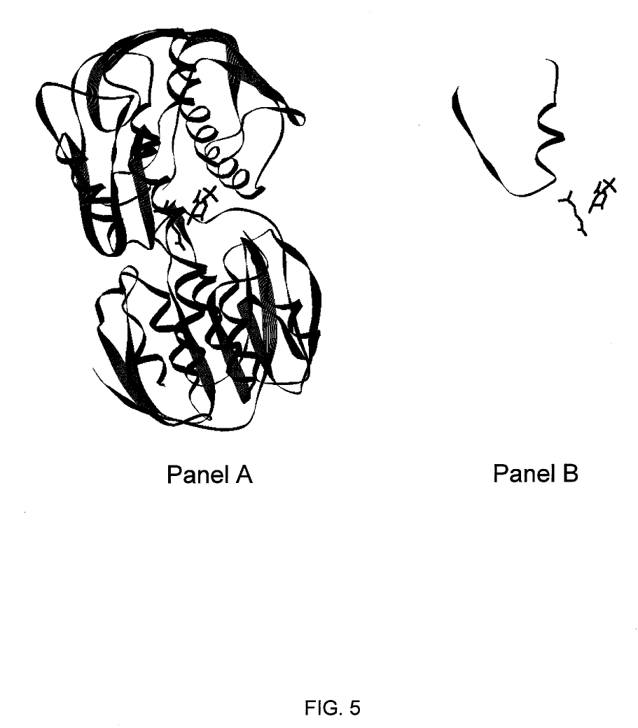

FIG. 6 shows a Western blot of leaf samples from transgenic maize plants expressing GRG1(EVO6), detected with a polyclonal antibody. Total protein was isolated from maize leaf samples, and expression of GRG1 (EVO6) protein was identified using Western blot analysis. Lane A shows 5 ng of purified GRG1(EVO6) protein, Lane B shows 1 ng of GRG1 protein. Lanes B through J show independent transgenic plants expressing grg1 (evo6). Lanes containing negative control plants show no signal.

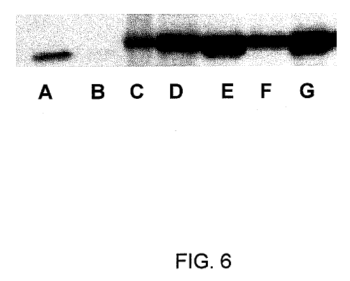

### DETAILED DESCRIPTION OF THE INVENTION

Compositions

Polypeptide sequences capable of conferring glyphosate tolerance or resistance are provided. The compositions include EPSP synthase polypeptides having the sequence domain X-C-X-E-S-G-L-S-X-R-X-F-X-P-X (SEQ ID NO:44), where X denotes any amino acid, the sequence domain D-C-X₁-X₂-S-G (SEQ ID NO:76), wherein X₁ denotes glutamine, valine, proline, glutamic acid, isoleucine, methionine, or threonine and X₂ denotes any amino acid, or the sequence domain X₁-C-X₂-E-S-G-L-S-X₃-R-X₄-F-X₅-P-X₆ (SEQ ID NO:45), and wherein X₁ denotes aspartic acid, lysine, glutamic acid, asparagine, serine, glycine, proline or arginine; X₂ denotes asparagine, alanine, serine, glycine, glutamine, valine, proline, glutamic acid, isoleucine, methionine, threonine, or arginine; where X₃ denotes isoleucine, methionine, phenylalanine, glycine, serine, or valine; where X₄ denotes methionine, alanine, serine, glycine, glutamine, leucine, valine, or isoleucine; where X₅ denotes threonine, alanine, valine, isoleucine, proline, leucine, or glycine; and where X₆ denotes isoleucine, leucine, cysteine, alanine, phenylalanine or methionine. This domain is located in the Q-loop region of the EPSP synthase polypeptide. The region (herein referred to as the “Q-loop”) corresponding to amino acids 80-105 of the glyphosate resistant EPSP synthase polypeptide GRG1 (SEQ ID NO:2; U.S. patent application Ser. No. 10/739,610) is known to be involved in the recognition of the EPSP synthase substrate phosphoenoylpyruvate (PEP) (Schönbrunn et al. (2001) Proc. Natl. Acad. Sci. USA 90:1376-1380, Stauffer et al. (2001) Biochemistry 40:3951-3957).

In one embodiment, this sequence domain corresponds to amino acid positions 85 through 99 of SEQ ID NO:2 and is selected from the group consisting of the corresponding positions of SEQ ID NO:5-43 and SEQ ID NO:56-65. In another embodiment, the polynucleotide comprising the sequence domain of the invention encodes an EPSP synthase polypeptide other than SEQ ID NO: 1 and 46 having at least 70% sequence identity to the amino acids corresponding to positions 1 through 84 and positions 100 through 431 of SEQ ID NO:2. As used herein, the phrase “corresponding to” or “corresponds to” when referring to amino acid (or nucleotide) position numbers means that one or more amino acid (or nucleotide) sequences aligns with the reference sequence at the position numbers specified in the reference sequence. For example, to identify a Q-loop region in an amino acid sequence that corresponds to amino acids 80-105 of SEQ ID NO:2, one could align the amino acid sequence in question with the amino acid sequence of SEQ ID NO:2 using alignment methods discussed elsewhere herein, and identify the region of the amino acid sequence in question that aligns with amino acid residues 80-105 of SEQ ID NO:2. It is recognized that the amino acid position designating the Q-loop may vary by about plus or minus 20, 15, 10, 9, 8, 7, 6, 5, 4, 3, 2 or 1 amino acid(s) on either side of the amino acids corresponding to positions 80-105 of SEQ ID NO:2.

The phrase “other than SEQ ID NO: 1 and 46” includes fragments of SEQ ID NO:1 or 46, including fragments comprising at least about 340, at least about 350, at least 400, at least 401, 402, 403, 404, 405, 406, 407, 408, 409, 410, 411, 412, 413, 414, 415, 416, 417, 418, 419, 420, 421, 422, 423, 424, 425, 426, 427, 428, 429, or 431 consecutive amino acids of SEQ ID NO:1 or 46. Additionally, it is recognized that sequences may be available in the prior art that contain a domain of the invention (see, for example, U.S. Patent Application Publication Nos. 20060143727, 20030049814, 20030079246, 20030200560, 2004148650, and 20050223436; U.S. Pat. Nos. 5,188,642, 6,040,497, 7,214,535, 7,169,970, 6,867,293, 7,183,110, 5,094,945, 6,225,114, 7,141,722, 7,045,684, 5,312,910, 6,566,587, and RE037,287, each of which is herein incorporated by reference in its entirety). Until the present invention such sequences may not be recognized in the art as having the ability to confer glyphosate resistance and those sequences are excluded from the compositions of the invention. To the extent those sequences were not known to confer resistance, they are included within the method claims. The methods of the invention provide a novel approach to identify and use sequences that have the ability to confer glyphosate resistance.

The EPSP synthase Q loop forms a portion of the binding pocket for PEP and glyphosate, and contains an invariant arginine that is known to hydrogen bond directly with the phosphate of PEP (Shuttleworth et al. (1999) Biochemistry 38:296-302). This Q-loop domain has been described as a predictor for glyphosate resistance and key residues within this domain have been identified (U.S. Application No. 60/658,320, herein incorporated by reference in its entirety). The compositions of the present invention include variants of GRG1 that exhibit either (1) continued ability to tolerate glyphosate, or (2) enhanced ability to tolerate glyphosate. Thus, the amino acid sequences of these variants expand and refine the key domains of the Q-loop for glyphosate resistance EPSP synthases.

A. Isolated Polynucleotides, and Variants and Fragments Thereof.

In some embodiments, the present invention comprises isolated or recombinant polynucleotides. An “isolated” or “purified” polynucleotide or polypeptide, or biologically active portion thereof, is substantially free of other cellular material, or culture medium when produced by recombinant techniques, or substantially free of chemical precursors or other chemicals when chemically synthesized. By “biologically active” is intended to possess the desired biological activity of the native polypeptide, that is, retain herbicide resistance activity. An “isolated” or “recombinant” polynucleotide may be free of sequences (for example, protein encoding sequences) that naturally flank the nucleic acid (i.e., sequences located at the 5′ and 3′ ends of the nucleic acid) in the genomic DNA of the organism from which the polynucleotide is derived. For purposes of the invention, “isolated” when used to refer to polynucleotides excludes isolated chromosomes. For example, in various embodiments, the isolated glyphosate resistance-encoding polynucleotide can contain less than about 5 kb, 4 kb, 3 kb, 2 kb, 1 kb, 0.5 kb, or 0.1 kb of nucleotide sequence that naturally flanks the polynucleotide in genomic DNA of the cell from which the polynucleotide is derived.

Polynucleotides of the invention include those encoding EPSP synthase polypeptides characterized by having the sequence domain of the invention. The information used in identifying the domains of the invention includes sequence alignments of EPSP synthase enzymes as described elsewhere herein. The sequence alignments are used to identify regions of homology between the sequences and to identify the domains that are characteristic of these EPSP synthase enzymes. In some embodiments, the domains of the invention are used to identify EPSP synthase enzymes that are glyphosate resistant. Further embodiments include polynucleotides that encode glyphosate-resistant polypeptides comprising SEQ ID NO:5-43 and SEQ ID NO:56-65, the polynucleotide sequences of SEQ ID NO:3, 4, 66, 67, 74, and 75, and polynucleotide sequences comprising SEQ ID NO:68-73.

By “glyphosate” is intended any herbicidal form of N-phosphonomethylglycine (including any salt thereof) and other forms that result in the production of the glyphosate anion in planta. An “herbicide resistance protein” or a protein resulting from expression of an “herbicide resistance-encoding nucleic acid molecule” includes proteins that confer upon a cell the ability to tolerate a higher concentration of an herbicide than cells that do not express the protein, or to tolerate a certain concentration of an herbicide for a longer time than cells that do not express the protein. A “glyphosate resistance protein” includes a protein that confers upon a cell the ability to tolerate a higher concentration of glyphosate than cells that do not express the protein, or to tolerate a certain concentration of glyphosate for a longer period of time than cells that do not express the protein. By “tolerate” or “tolerance” is intended either to survive, or to carry out essential cellular functions such as protein synthesis and respiration in a manner that is not readily discernable from untreated cells.

The present invention further contemplates variants and fragments of the polynucleotides described herein. A “fragment” of a polynucleotide may encode a biologically active portion of a polypeptide, or it may be a fragment that can be used as a hybridization probe or PCR primer using methods disclosed elsewhere herein. Polynucleotides that are fragments of a polynucleotide comprise at least about 15, 20, 50, 75, 100, 200, 300, 350, 400, 450, 500, 550, 600, 650, 700, 750, 800, 850, 900, 950, 1000, 1050, 1100, 1150, 1200, 1250, 1300, 1350, 1400, 1450, 1500, 1550, 1600, 1650, 1700, 1750, 1800, 1850, 1900, 1950 contiguous nucleotides, or up to the number of nucleotides present in a full-length polynucleotide disclosed herein depending upon the intended use (e.g., an EPSP synthase polynucleotide comprising SEQ ID NO: 1). By “contiguous” nucleotides is intended nucleotide residues that are immediately adjacent to one another.

Fragments of the polynucleotides of the present invention generally will encode polypeptide fragments that retain the biological activity of the full-length glyphosate resistance protein; i.e., herbicide-resistance activity. By “retains herbicide resistance activity” is intended that the fragment will have at least about 30%, at least about 50%, at least about 70%, at least about 80%, 85%, 90%, 95%, 100%, 110%, 125%, 150%, 175%, 200%, 250%, at least about 300% or greater of the herbicide resistance activity of the full-length glyphosate resistance protein disclosed herein as SEQ ID NO:2. Methods for measuring herbicide resistance activity are well known in the art. See, for example, U.S. Pat. Nos. 4,535,060, and 5,188,642, each of which are herein incorporated by reference in their entirety.

A fragment of a polynucleotide that encodes a biologically active portion of a polypeptide of the invention will encode at least about 15, 25, 30, 50, 75, 100, 125, 150, 175, 200, 250, 300, 350, 400 contiguous amino acids, or up to the total number of amino acids present in a full-length polypeptide of the invention.

Preferred herbicide resistance proteins of the present invention are encoded by a nucleotide sequence comprising a polynucleotide encoding a polypeptide having a sequence domain disclosed herein. In one embodiment, this sequence domain corresponds to amino acid positions 85 through 99 of SEQ ID NO:2 and is selected from the group consisting of the corresponding positions of SEQ ID NO:5-43 and SEQ ID NO:56-65. In another embodiment, the polynucleotide comprising the sequence domain of the invention encodes an EPSP synthase that is sufficiently identical at the amino acids corresponding to positions 1 through 84 and positions 100 through 431 of SEQ ID NO:2. The term “sufficiently identical” is intended an amino acid or nucleotide sequence that has at least about 60% or 65% sequence identity, about 70% or 75% sequence identity, about 80% or 85% sequence identity, or about 90%, 91%, 92%, 93%, 94%, 95%, 96%, 97%, 98% or 99% sequence identity compared to a reference sequence using one of the alignment programs described herein using standard parameters. In another embodiment, the polynucleotide comprising the domain of the invention encodes an EPSP synthase that has one or more additions, substitutions or deletions in the region corresponding to amino acid positions 1 through 84 and positions 100 through 431 of SEQ ID NO:2, up to about 2, about 3, about 4, about 5, about 6, about 7, about 8, about 9, about 10, about 15, about 20, about 25, about 30, about 35, about 40, about 45, about 50, about 55, about 60, about 65, about 70, about 75, about 80, about 85, about 90, about 100 or more amino acid substitutions, deletions or insertions. One of skill in the art will recognize that these values can be appropriately adjusted to determine corresponding identity of proteins encoded by two nucleotide sequences by taking into account codon degeneracy, amino acid similarity, reading frame positioning, and the like.

To determine the percent identity of two amino acid sequences or of two nucleic acids, the sequences are aligned for optimal comparison purposes. The percent identity between the two sequences is a function of the number of identical positions shared by the sequences (i.e., percent identity=number of identical positions/total number of positions (e.g., overlapping positions)×100). In one embodiment, the two sequences are the same length. The percent identity between two sequences can be determined using techniques similar to those described below, with or without allowing gaps. In calculating percent identity, typically exact matches are counted. For the purposes of the present invention, when calculating percent identity in the regions corresponding to amino acid positions 1 through 84 and positions 100 through 431, the percent across the entire region (1 through 84 plus 100 through 431) is measured.

The determination of percent identity between two sequences can be accomplished using a mathematical algorithm. A nonlimiting example of a mathematical algorithm utilized for the comparison of two sequences is the algorithm of Karlin and Altschul (1990) Proc. Natl. Acad. Sci. USA 87:2264, modified as in Karlin and Altschul (1993) Proc. Natl. Acad. Sci. USA 90:5873-5877. Such an algorithm is incorporated into the BLASTN and BLASTX programs of Altschul et al. (1990) J. Mol. Biol. 215:403. BLAST nucleotide searches can be performed with the BLASTN program, score=100, wordlength=12, to obtain nucleotide sequences homologous to glyphosate-resistant nucleic acid molecules of the invention. BLAST protein searches can be performed with the BLASTX program, score=50, wordlength=3, to obtain amino acid sequences homologous to herbicide resistance protein molecules of the invention. To obtain gapped alignments for comparison purposes, Gapped BLAST can be utilized as described in Altschul et al. (1997) Nucleic Acids Res. 25:3389. Alternatively, PSI-Blast can be used to perform an iterated search that detects distant relationships between molecules. See Altschul et al. (1997) supra. When utilizing BLAST, Gapped BLAST, and PSI-Blast programs, the default parameters of the respective programs (e.g., BLASTX and BLASTN) can be used. See www.ncbi.nlm.nih.gov. Another non-limiting example of a mathematical algorithm utilized for the comparison of sequences is the ClustalW algorithm (Higgins et al. (1994) Nucleic Acids Res. 22:4673-4680). ClustalW compares sequences and aligns the entirety of the amino acid or DNA sequence, and thus can provide data about the sequence conservation of the entire amino acid sequence. The ClustalW algorithm is used in several commercially available DNA/amino acid analysis software packages, such as the ALIGNX module of the Vector NTI Program Suite (Invitrogen Corporation, Carlsbad, Calif.). After alignment of amino acid sequences with ClustalW, the percent amino acid identity can be assessed. A non-limiting example of a software program useful for analysis of ClustalW alignments is GENEDOC™. GENEDOC™ (Karl Nicholas) allows assessment of amino acid (or DNA) similarity and identity between multiple proteins. Another non-limiting example of a mathematical algorithm utilized for the comparison of sequences is the algorithm of Myers and Miller (1988) CABIOS 4:11-17. Such an algorithm is incorporated into the ALIGN program (version 2.0), which is part of the GCG sequence alignment software package (available from Accelrys, Inc., 9865 Scranton Rd., San Diego, Calif., USA). When utilizing the ALIGN program for comparing amino acid sequences, a PAM120 weight residue table, a gap length penalty of 12, and a gap penalty of 4 can be used.

Unless otherwise stated, GAP Version 10, which uses the algorithm of Needleman and Wunsch (1970) supra, will be used to determine sequence identity or similarity using the following parameters: % identity and % similarity for a nucleotide sequence using GAP Weight of 50 and Length Weight of 3, and the nwsgapdna.cmp scoring matrix; % identity or % similarity for an amino acid sequence using GAP weight of 8 and length weight of 2, and the BLOSUM62 scoring program. Equivalent programs may also be used. By “equivalent program” is intended any sequence comparison program that, for any two sequences in question, generates an alignment having identical nucleotide residue matches and an identical percent sequence identity when compared to the corresponding alignment generated by GAP Version 10.

The invention also encompasses variant polynucleotides. “Variants” of the polynucleotide include those sequences that encode the polypeptides disclosed herein but that differ conservatively because of the degeneracy of the genetic code, as well as those that are sufficiently identical.

Bacterial genes quite often possess multiple methionine initiation codons in proximity to the start of the open reading frame. Often, translation initiation at one or more of these start codons will lead to generation of a functional protein. These start codons can include ATG codons. However, bacteria such as Bacillus sp. also recognize the codon GTG as a start codon, and proteins that initiate translation at GTG codons contain a methionine at the first amino acid. Furthermore, it is not often determined a priori which of these codons are used naturally in the bacterium. Thus, it is understood that use of one of the alternate methionine codons may lead to generation of variants that confer herbicide resistance. These herbicide resistance proteins are encompassed in the present invention and may be used in the methods of the present invention.

Naturally occurring allelic variants can be identified with the use of well-known molecular biology techniques, such as polymerase chain reaction (PCR) and hybridization techniques as outlined below. Variant polynucleotides also include synthetically derived polynucleotides that have been generated, for example, by using site-directed or other mutagenesis strategies but which still encode the polypeptide having the desired biological activity.

The skilled artisan will further appreciate that changes can be introduced by further mutation of the polynucleotides of the invention thereby leading to further changes in the amino acid sequence of the encoded polypeptides, without altering the biological activity of the polypeptides. Thus, variant isolated polynucleotides can be created by introducing one or more additional nucleotide substitutions, additions, or deletions into the corresponding polynucleotide encoding the EPSP synthase domain disclosed herein, such that one or more amino acid substitutions, additions or deletions are introduced into the encoded polypeptide. Further mutations can be introduced by standard techniques, such as site-directed mutagenesis and PCR-mediated mutagenesis, or gene shuffling techniques. Such variant polynucleotides are also encompassed by the present invention.

Variant polynucleotides can be made by introducing mutations randomly along all or part of the coding sequence, such as by saturation mutagenesis, and the resultant mutants can be screened for the ability to confer herbicide resistance activity to identify mutants that retain activity.

Gene shuffling or sexual PCR procedures (for example, Smith (1994) Nature 370:324-325; U.S. Pat. Nos. 5,837,458; 5,830,721; 5,811,238; and 5,733,731, each of which is herein incorporated by reference) can be used to further modify or enhance polynucleotides and polypeptides having the EPSP synthase domain of the present invention (for example, polypeptides that confer glyphosate resistance). Gene shuffling involves random fragmentation of several mutant DNAs followed by their reassembly by PCR into full length molecules. Examples of various gene shuffling procedures include, but are not limited to, assembly following DNase treatment, the staggered extension process (STEP), and random priming in vitro recombination. In the DNase mediated method, DNA segments isolated from a pool of positive mutants are cleaved into random fragments with DNaseI and subjected to multiple rounds of PCR with no added primer. The lengths of random fragments approach that of the uncleaved segment as the PCR cycles proceed, resulting in mutations in different clones becoming mixed and accumulating in some of the resulting sequences. Multiple cycles of selection and shuffling have led to the functional enhancement of several enzymes (Stemmer (1994) Nature 370:389-391; Stemmer (1994) Proc. Natl. Acad. Sci. USA 91:10747-10751; Crameri et al. (1996) Nat. Biotechnol. 14:315-319; Zhang et al. (1997) Proc. Natl. Acad. Sci. USA 94:4504-4509; and Crameri et al. (1997) Nat. Biotechnol. 15:436-438). Such procedures could be performed, for example, on polynucleotides encoding polypeptides having the sequence domain of the present invention to generate polypeptides that confer glyphosate resistance.

Using methods such as PCR, hybridization, and the like corresponding herbicide resistance sequences can be identified by looking for EPSP synthase domains of the present invention. See, for example, Sambrook and Russell (2001) Molecular Cloning: A Laboratory Manual (Cold Spring Harbor Laboratory Press, Cold Spring Harbor, N.Y.) and Innis et al. (1990) PCR Protocols: A Guide to Methods and Applications (Academic Press, NY).

In a hybridization method, all or part of the herbicide resistance nucleotide sequence can be used to screen cDNA or genomic libraries. Methods for construction of such cDNA and genomic libraries are generally known in the art and are disclosed in Sambrook and Russell, 2001, supra. The so-called hybridization probes may be genomic DNA fragments, cDNA fragments, RNA fragments, or other oligonucleotides, and may be labeled with a detectable group such as ³²P, or any other detectable marker, such as other radioisotopes, a fluorescent compound, an enzyme, or an enzyme co-factor. Probes for hybridization can be made by labeling synthetic oligonucleotides based on the known herbicide resistance-encoding nucleotide sequences disclosed herein. Degenerate primers designed on the basis of conserved nucleotides or amino acid residues in the nucleotide sequences or encoded amino acid sequences can additionally be used. The probe typically comprises a region of nucleotide sequence that hybridizes under stringent conditions to at least about 12, preferably about 25, at least about 50, 75, 100, 125, 150, 175, 200, 250, 300, 350, 400, 500, 600, 700, 800, 900, 1000, 1200, 1300 consecutive nucleotides of an herbicide resistance-encoding nucleotide sequence of the invention or a fragment or variant thereof. Methods for the preparation of probes for hybridization are generally known in the art and are disclosed in Sambrook and Russell, 2001, supra and Sambrook et al. (1989) Molecular Cloning: A Laboratory Manual (2d ed., Cold Spring Harbor Laboratory Press, Cold Spring Harbor, N.Y.), both of which are herein incorporated by reference.

For example, an entire herbicide resistance sequence disclosed herein, or one or more portions thereof, may be used as a probe capable of specifically hybridizing to corresponding herbicide resistance sequences and messenger RNAs. To achieve specific hybridization under a variety of conditions, such probes include sequences that are unique and are at least about 10 nucleotides in length, or at least about 20 nucleotides in length. Such probes may be used to amplify corresponding herbicide resistance sequences from a chosen organism by PCR. This technique may be used to isolate additional coding sequences from a desired organism or as a diagnostic assay to determine the presence of coding sequences in an organism. Hybridization techniques include hybridization screening of plated DNA libraries (either plaques or colonies; see, for example, Sambrook et al. (1989) Molecular Cloning: A Laboratory Manual (2d ed., Cold Spring Harbor Laboratory Press, Cold Spring Harbor, N.Y.).

Hybridization of such sequences may be carried out under stringent conditions. By “stringent conditions” or “stringent hybridization conditions” is intended conditions under which a probe will hybridize to its target sequence to a detectably greater degree than to other sequences (e.g., at least 2-fold over background). Stringent conditions are sequence-dependent and will be different in different circumstances. By controlling the stringency of the hybridization and/or washing conditions, target sequences that are 100% complementary to the probe can be identified (homologous probing). Alternatively, stringency conditions can be adjusted to allow some mismatching in sequences so that lower degrees of similarity are detected (heterologous probing). Generally, a probe is less than about 1000 nucleotides in length, preferably less than 500 nucleotides in length.

Typically, stringent conditions will be those in which the salt concentration is less than about 1.5 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. Exemplary low stringency conditions include hybridization with a buffer solution of 30 to 35% formamide, 1 M NaCl, 1% SDS (sodium dodecyl sulfate) at 37° C., and a wash in 1× to 2×SSC (20×SSC=3.0 M NaCl/0.3 M trisodium citrate) at 50 to 55° C. Exemplary moderate stringency conditions include hybridization in 40 to 45% formamide, 1.0 M NaCl, 1% SDS at 37° C., and a wash in 0.5× to 1×SSC at 55 to 60° C. Exemplary high stringency conditions include hybridization in 50% formamide, 1 M NaCl, 1% SDS at 37° C., and a wash in 0.1×SSC at 60 to 65° C. Optionally, wash buffers may comprise about 0.1% to about 1% SDS. Duration of hybridization is generally less than about 24 hours, usually about 4 to about 12 hours.

Specificity is typically the function of post-hybridization washes, the critical factors being the ionic strength and temperature of the final wash solution. For DNA-DNA hybrids, the Tₘ can be approximated from the equation of Meinkoth and Wahl (1984) Anal. Biochem. 138:267-284: Tₘ=81.5° C.+16.6 (log M)+0.41 (% GC)-0.61 (% form)-500/L; where M is the molarity of monovalent cations, % GC is the percentage of guanosine and cytosine nucleotides in the DNA, % form is the percentage of formamide in the hybridization solution, and L is the length of the hybrid in base pairs. The Tₘ is the temperature (under defined ionic strength and pH) at which 50% of a complementary target sequence hybridizes to a perfectly matched probe. Tₘ is reduced by about 1° C. for each 1% of mismatching; thus, Tₘ, hybridization, and/or wash conditions can be adjusted to hybridize to sequences of the desired identity. For example, if sequences with ≧90% identity are sought, the Tₘ can be decreased 10° C. Generally, stringent conditions are selected to be about 5° C. lower than the thermal melting point (Tₘ) for the specific sequence and its complement at a defined ionic strength and pH. However, severely stringent conditions can utilize a hybridization and/or wash at 1, 2, 3, or 4° C. lower than the thermal melting point (Tₘ); moderately stringent conditions can utilize a hybridization and/or wash at 6, 7, 8, 9, or 10° C. lower than the thermal melting point (Tₘ); low stringency conditions can utilize a hybridization and/or wash at 11, 12, 13, 14, 15, or 20° C. lower than the thermal melting point (Tₘ). Using the equation, hybridization and wash compositions, and desired Tₘ, those of ordinary skill will understand that variations in the stringency of hybridization and/or wash solutions are inherently described. If the desired degree of mismatching results in a Tₘ of less than 45° C. (aqueous solution) or 32° C. (formamide solution), it is preferred to increase the SSC concentration so that a higher temperature can be used. An extensive guide to the hybridization of nucleic acids is found in Tijssen (1993) Laboratory Techniques in Biochemistry and Molecular Biology—Hybridization with Nucleic Acid Probes, Part I, Chapter 2 (Elsevier, New York); and Ausubel et al., eds. (1995) Current Protocols in Molecular Biology, Chapter 2 (Greene Publishing and Wiley-Interscience, New York). See Sambrook et al. (1989) Molecular Cloning: A Laboratory Manual (2d ed., Cold Spring Harbor Laboratory Press, Cold Spring Harbor, N.Y.).

B. Isolated Proteins and Variants and Fragments Thereof

Herbicide resistance polypeptides are also encompassed within the present invention. An herbicide resistance polypeptide that is substantially free of cellular material includes preparations of polypeptides having less than about 30%, 20%, 10%, or 5% (by dry weight) of non-herbicide resistance polypeptide (also referred to herein as a “contaminating protein”). In the present invention, “herbicide resistance protein” is intended an EPSP synthase polypeptide having the sequence domain of the invention. Fragments, biologically active portions, and variants thereof are also provided, and may be used to practice the methods of the present invention.

“Fragments” or “biologically active portions” include polypeptide fragments comprising a portion of an amino acid sequence encoding an herbicide resistance protein and that retains herbicide resistance activity. A biologically active portion of an herbicide resistance protein can be a polypeptide that is, for example, 10, 25, 50, 100 or more amino acids in length. This protein may be altered in various ways including amino acid substitutions, deletions, truncations, and insertions of one or more amino acids of in the region corresponding to amino acid positions 85 through 99 of SEQ ID NO:2, including up to about 2, about 3, about 4, about 5, about 6, about 7, about 8, about 9, about 10, about 15, about 20, about 25, about 30, about 35, about 40, about 45, about 50, about 55, about 60, about 65, about 70, about 75, about 80, about 85, about 90, about 100 or more amino acid substitutions, deletions or insertions. Such biologically active portions can be prepared by recombinant techniques and evaluated for herbicide resistance activity. Methods for measuring herbicide resistance activity are well known in the art. See, for example, U.S. Pat. Nos. 4,535,060, and 5,188,642, each of which are herein incorporated by reference in their entirety. As used here, a fragment comprises at least 8 contiguous amino acids of SEQ ID NO:5-43 or 56-65. The invention encompasses other fragments, however, such as any fragment in the protein greater than about 10, 20, 30, 50, 100, 150, 200, 250, 300, 350, or 400 amino acids.

By “variants” is intended proteins or polypeptides having an amino acid sequence that is at least about 60%, 65%, about 70%, 75%, about 80%, 85%, 90%, 91%, 92%, 93%, 94%, 95%, 96%, 97%, 98% or 99% identical to an EPSP synthase polypeptide having the EPSP synthase domain of the present invention. One of skill in the art will recognize that these values can be appropriately adjusted to determine corresponding identity of polypeptides encoded by two polynucleotides by taking into account codon degeneracy, amino acid similarity, reading frame positioning, and the like.

For example, conservative amino acid substitutions may be made at one or more nonessential amino acid residues. A “nonessential” amino acid residue is a residue that can be altered from the wild-type sequence of a polypeptide without altering the biological activity, whereas an “essential” amino acid residue is required for biological activity. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Amino acid substitutions may be made in nonconserved regions that retain function. In general, such substitutions would not be made for conserved amino acid residues, or for amino acid residues residing within a conserved motif, where such residues are essential for polypeptide activity. However, one of skill in the art would understand that functional variants may have minor conserved or nonconserved alterations in the conserved residues.

Antibodies to the polypeptides of the present invention, or to variants or fragments thereof, are also encompassed. Methods for producing antibodies are well known in the art (see, for example, Harlow and Lane (1988) Antibodies: A Laboratory Manual, Cold Spring Harbor Laboratory, Cold Spring Harbor, N.Y.; U.S. Pat. No. 4,196,265).

In one embodiment of the present invention, the glyphosate-resistant EPSPS enzyme has a Kₘ for phosphoenolpyruvate (PEP) between about 1 and about 150 uM, including about 2 uM, about 3, 4, 5, 6, 7, 8, 9, 10, 20, 30, 40, 50, 60, 70, 80, 90, 100, 110, 120, 130 or about 140 uM, and a Kᵢ (glyphosate)/Kₘ (PEP) between about 50 and about 2000, between about 100 and about 1000, about 150, about 200, about 250, about 300, about 350, about 400, about 450, about 500, about 600, about 700, about 800, about 900, about 1000, about 1100, about 1200, about 1300, about 1400, about 1500, about 1600, about 1700, about 1800, about 1900, or up to about 2000. As used herein, Kₘ and Kᵢ are measured under conditions in which the enzyme obeys Michaelis-Menten kinetics, around pH 7. One nonlimiting measurement technique uses the enzyme in purified form in potassium chloride and HEPES buffer at pH 7 at room temperature and uses concentrations of glyphosate from 0 to 110 mM.

EPSP synthase kinetic activity can be assayed, for example, by measuring the liberation of phosphate that results during the catalysis of a substrate of EPSP synthase (for example, PEP and S3P) to its subsequent reaction product (for example, 5-enolpyruvyl-3-phosphoshikimic acid) using a fluorescent assay described by Vazquez et al. (2003) Anal. Biochem. 320(2):292-298 and in U.S. patent application Ser. No. 11/605,824 entitled “grg23 and grg51 Genes Conferring Herbicide Resistance,” filed Nov. 29, 2006 and herein incorporated by reference in its entirety.

C. Polynucleotide Constructs

The polynucleotides encoding the EPSP synthase domain of the present invention may be modified to obtain or enhance expression in plant cells. The polynucleotides encoding the polypeptides identified by the methods of the invention may be provided in expression cassettes for expression in the plant of interest. A “plant expression cassette” includes a DNA construct, such as a recombinant DNA construct, that is capable of resulting in the expression of a polynucleotide in a plant cell. The cassette can include in the 5′-3′ direction of transcription, a transcriptional initiation region (i.e., promoter) operably-linked to one or more polynucleotides of interest, and a translation and/or transcriptional termination region (i.e., termination region) functional in plants. The cassette may additionally contain at least one additional polynucleotide to be introduced into the organism, such as a selectable marker gene. Alternatively, the additional polynucleotide(s) can be provided on multiple expression cassettes. Such an expression cassette is provided with a plurality of restriction sites for insertion of the polynucleotide(s) to be under the transcriptional regulation of the regulatory regions.

“Heterologous” generally refers to the polynucleotide or polypeptide that is not endogenous to the cell or is not endogenous to the location in the native genome in which it is present, and has been added to the cell by infection, transfection, microinjection, electroporation, microprojection, or the like. By “operably linked” is intended a functional linkage between two polynucleotides. For example, when a promoter is operably linked to a DNA sequence, the promoter sequence initiates and mediates transcription of the DNA sequence. It is recognized that operably linked polynucleotides may or may not be contiguous and, where used to reference the joining of two polypeptide coding regions, the polypeptides are expressed in the same reading frame.

The promoter may be any polynucleotide sequence which shows transcriptional activity in the chosen plant cells, plant parts, or plants. The promoter may be native or analogous, or foreign or heterologous, to the plant host and/or to the DNA sequence of the invention. Where the promoter is “native” or “analogous” to the plant host, it is intended that the promoter is found in the native plant into which the promoter is introduced. Where the promoter is “foreign” or “heterologous” to the DNA sequence of the invention, it is intended that the promoter is not the native or naturally occurring promoter for the operably linked DNA sequence of the invention. The promoter may be inducible or constitutive. It may be naturally-occurring, may be composed of portions of various naturally-occurring promoters, or may be partially or totally synthetic. Guidance for the design of promoters is provided by studies of promoter structure, such as that of Harley and Reynolds (1987) Nucleic Acids Res. 15:2343-2361. Also, the location of the promoter relative to the transcription start may be optimized. See, e.g., Roberts et al. (1979) Proc. Natl. Acad. Sci. USA, 76:760-764. Many suitable promoters for use in plants are well known in the art.

For instance, suitable constitutive promoters for use in plants include: the promoters from plant viruses, such as the peanut chlorotic streak caulimovirus (PClSV) promoter (U.S. Pat. No. 5,850,019); the 35S promoter from cauliflower mosaic virus (CaMV) (Odell et al. (1985) Nature 313:810-812); promoters of Chlorella virus methyltransferase genes (U.S. Pat. No. 5,563,328) and the full-length transcript promoter from figwort mosaic virus (FMV) (U.S. Pat. No. 5,378,619); the promoters from such genes as rice actin (McElroy et al. (1990) Plant Cell 2:163-171); ubiquitin (Christensen et al. (1989) Plant Mol. Biol. 12:619-632 and Christensen et al. (1992) Plant Mol. Biol. 18:675-689); pEMU (Last et al. (1991) Theor. Appl. Genet. 81:581-588); MAS (Velten et al. (1984) EMBO J. 3:2723-2730); maize H3 histone (Lepetit et al. (1992) Mol. Gen. Genet. 231:276-285 and Atanassova et al. (1992) Plant J. 2(3):291-300); Brassica napus ALS3 (PCT application WO 97/41228); and promoters of various Agrobacterium genes (see U.S. Pat. Nos. 4,771,002; 5,102,796; 5,182,200; and 5,428,147).

Suitable inducible promoters for use in plants include: the promoter from the ACE1 system which responds to copper (Mett et al. (1993) PNAS 90:4567-4571); the promoter of the maize In2 gene which responds to benzenesulfonamide herbicide safeners (Hershey et al. (1991) Mol. Gen. Genetics 227:229-237 and Gatz et al. (1994) Mol. Gen. Genetics 243:32-38); and the promoter of the Tet repressor from Tn10 (Gatz et al. (1991) Mol. Gen. Genet. 227:229-237). Another inducible promoter for use in plants is one that responds to an inducing agent to which plants do not normally respond. An exemplary inducible promoter of this type is the inducible promoter from a steroid hormone gene, the transcriptional activity of which is induced by a glucocorticosteroid hormone (Schena et al. (1991) Proc. Natl. Acad. Sci. USA 88:10421) or the recent application of a chimeric transcription activator, XVE, for use in an estrogen receptor-based inducible plant expression system activated by estradiol (Zuo et al. (2000) Plant J, 24:265-273). Other inducible promoters for use in plants are described in EP 332104, PCT WO 93/21334 and PCT WO 97/06269 which are herein incorporated by reference in their entirety. Promoters composed of portions of other promoters and partially or totally synthetic promoters can also be used. See, e.g., Ni et al. (1995) Plant J. 7:661-676 and PCT WO 95/14098 describing such promoters for use in plants.

The promoter may include, or be modified to include, one or more enhancer elements. In some embodiments, the promoter may include a plurality of enhancer elements. Promoters containing enhancer elements provide for higher levels of transcription as compared to promoters that do not include them. Suitable enhancer elements for use in plants include the PClSV enhancer element (U.S. Pat. No. 5,850,019), the CaMV 35S enhancer element (U.S. Pat. Nos. 5,106,739 and 5,164,316) and the FMV enhancer element (Maiti et al. (1997) Transgenic Res. 6:143-156). See also PCT WO 96/23898.

Often, such constructs can contain 5′ and 3′ untranslated regions. Such constructs may contain a “signal sequence” or “leader sequence” to facilitate co-translational or post-translational transport of the peptide of interest to certain intracellular structures such as the chloroplast (or other plastid), endoplasmic reticulum, or Golgi apparatus, or to be secreted. For example, the construct can be engineered to contain a signal peptide to facilitate transfer of the peptide to the endoplasmic reticulum. By “signal sequence” is intended a sequence that is known or suspected to result in cotranslational or post-translational peptide transport across the cell membrane. In eukaryotes, this typically involves secretion into the Golgi apparatus, with some resulting glycosylation. By “leader sequence” is intended any sequence that, when translated, results in an amino acid sequence sufficient to trigger co-translational transport of the peptide chain to a sub-cellular organelle. Thus, this includes leader sequences targeting transport and/or glycosylation by passage into the endoplasmic reticulum, passage to vacuoles, plastids including chloroplasts, mitochondria, and the like. It may also be preferable to engineer the plant expression cassette to contain an intron, such that mRNA processing of the intron is required for expression.

By “3′ untranslated region” is intended a polynucleotide located downstream of a coding sequence. Polyadenylation signal sequences and other sequences encoding regulatory signals capable of affecting the addition of polyadenylic acid tracts to the 3′ end of the mRNA precursor are 3′ untranslated regions. By “5′ untranslated region” is intended a polynucleotide located upstream of a coding sequence.

Other upstream or downstream untranslated elements include enhancers. Enhancers are polynucleotides that act to increase the expression of a promoter region. Enhancers are well known in the art and include, but are not limited to, the SV40 enhancer region and the 35S enhancer element.

The termination region may be native with the transcriptional initiation region, may be native with the sequence of the present invention, or may be derived from another source. Convenient termination regions are available from the Ti-plasmid of A. tumefaciens, such as the octopine synthase and nopaline synthase termination regions. See also Guerineau et al. (1991) Mol. Gen. Genet. 262:141-144; Proudfoot (1991) Cell 64:671-674; Sanfacon et al. (1991) Genes Dev. 5:141-149; Mogen et al. (1990) Plant Cell 2:1261-1272; Munroe et al. (1990) Gene 91:151-158; Ballas et al. (1989) Nucleic Acids Res. 17:7891-7903; and Joshi et al. (1987) Nucleic Acid Res. 15:9627-9639.

In one aspect of the invention, synthetic DNA sequences are designed for a given polypeptide, such as the polypeptides of the invention. Expression of the open reading frame of the synthetic DNA sequence in a cell results in production of the polypeptide of the invention. Synthetic DNA sequences can be useful to simply remove unwanted restriction endonuclease sites, to facilitate DNA cloning strategies, to alter or remove any potential codon bias, to alter or improve GC content, to remove or alter alternate reading frames, and/or to alter or remove intron/exon splice recognition sites, polyadenylation sites, Shine-Delgarno sequences, unwanted promoter elements and the like that may be present in a native DNA sequence. It is also possible that synthetic DNA sequences may be utilized to introduce other improvements to a DNA sequence, such as introduction of an intron sequence, creation of a DNA sequence that in expressed as a protein fusion to organelle targeting sequences, such as chloroplast transit peptides, apoplast/vacuolar targeting peptides, or peptide sequences that result in retention of the resulting peptide in the endoplasmic reticulum. Synthetic genes can also be synthesized using host cell-preferred codons for improved expression, or may be synthesized using codons at a host-preferred codon usage frequency. See, for example, Campbell and Gowri (1990) Plant Physiol. 92:1-11; U.S. Pat. Nos. 6,320,100; 6,075,185; 5,380,831; and 5,436,391, U.S. Published Application Nos. 20040005600 and 20010003849, and Murray et al. (1989) Nucleic Acids Res. 17:477-498, herein incorporated by reference.

In one embodiment, the polynucleotides of interest are targeted to the chloroplast for expression. In this manner, where the polynucleotide of interest is not directly inserted into the chloroplast, the expression cassette will additionally contain a polynucleotide encoding a transit peptide to direct the nucleotide of interest to the chloroplasts. Such transit peptides are known in the art. See, for example, Von Heijne et al. (1991) Plant Mol. Biol. Rep. 9:104-126; Clark et al. (1989) J. Biol. Chem. 264:17544-17550; Della-Cioppa et al. (1987) Plant Physiol. 84:965-968; Romer et al. (1993) Biochem. Biophys. Res. Commun. 196:1414-1421; and Shah et al. (1986) Science 233:478-481.

The polynucleotides of interest to be targeted to the chloroplast may be optimized for expression in the chloroplast to account for differences in codon usage between the plant nucleus and this organelle. In this manner, the polynucleotides of interest may be synthesized using chloroplast-preferred codons. See, for example, U.S. Pat. No. 5,380,831, herein incorporated by reference.

This plant expression cassette can be inserted into a plant transformation vector. By “transformation vector” is intended a DNA molecule that allows for the transformation of a cell. Such a molecule may consist of one or more expression cassettes, and may be organized into more than one vector DNA molecule. For example, binary vectors are plant transformation vectors that utilize two non-contiguous DNA vectors to encode all requisite cis- and trans-acting functions for transformation of plant cells (Hellens and Mullineaux (2000) Trends in Plant Science 5:446-451). “Vector” refers to a polynucleotide construct designed for transfer between different host cells. “Expression vector” refers to a vector that has the ability to incorporate, integrate and express heterologous DNA sequences or fragments in a foreign cell.

The plant transformation vector comprises one or more DNA vectors for achieving plant transformation. For example, it is a common practice in the art to utilize plant transformation vectors that comprise more than one contiguous DNA segment. These vectors are often referred to in the art as binary vectors. Binary vectors as well as vectors with helper plasmids are most often used for Agrobacterium-mediated transformation, where the size and complexity of DNA segments needed to achieve efficient transformation is quite large, and it is advantageous to separate functions onto separate DNA molecules. Binary vectors typically contain a plasmid vector that contains the cis-acting sequences required for T-DNA transfer (such as left border and right border), a selectable marker that is engineered to be capable of expression in a plant cell, and a “polynucleotide of interest” (a polynucleotide engineered to be capable of expression in a plant cell for which generation of transgenic plants is desired). Also present on this plasmid vector are sequences required for bacterial replication. The cis-acting sequences are arranged in a fashion to allow efficient transfer into plant cells and expression therein. For example, the selectable marker sequence and the sequence of interest are located between the left and right borders. Often a second plasmid vector contains the trans-acting factors that mediate T-DNA transfer from Agrobacterium to plant cells. This plasmid often contains the virulence functions (Vir genes) that allow infection of plant cells by Agrobacterium, and transfer of DNA by cleavage at border sequences and vir-mediated DNA transfer, as is understood in the art (Hellens and Mullineaux (2000) Trends in Plant Science, 5:446-451). Several types of Agrobacterium strains (e.g., LBA4404, GV3101, EHA101, EHA105, etc.) can be used for plant transformation. The second plasmid vector is not necessary for introduction of polynucleotides into plants by other methods such as microprojection, microinjection, electroporation, polyethylene glycol, etc.

D. Plant Transformation

Methods of the invention involve introducing a nucleotide construct into a plant. By “introducing” is intended to present to the plant the nucleotide construct in such a manner that the construct gains access to the interior of a cell of the plant. The methods of the invention do not require that a particular method for introducing a nucleotide construct to a plant is used, only that the nucleotide construct gains access to the interior of at least one cell of the plant. Methods for introducing nucleotide constructs into plants are known in the art including, but not limited to, stable transformation methods, transient transformation methods, and virus-mediated methods.

In general, plant transformation methods involve transferring heterologous DNA into target plant cells (e.g. immature or mature embryos, suspension cultures, undifferentiated callus, protoplasts, etc.), followed by applying a maximum threshold level of appropriate selection (depending on the selectable marker gene and in this case “glyphosate”) to recover the transformed plant cells from a group of untransformed cell mass. Explants are typically transferred to a fresh supply of the same medium and cultured routinely. Subsequently, the transformed cells are differentiated into shoots after placing on regeneration medium supplemented with a maximum threshold level of selecting agent (e.g. “glyphosate”). The shoots are then transferred to a selective rooting medium for recovering rooted shoot or plantlet. The transgenic plantlet then grow into mature plants and produce fertile seeds (e.g. Hiei et al. (1994) The Plant Journal 6:271-282; Ishida et al. (1996) Nature Biotechnology 14:745-750). Explants are typically transferred to a fresh supply of the same medium and cultured routinely. A general description of the techniques and methods for generating transgenic plants are found in Ayres and Park (1994) Critical Reviews in Plant Science 13:219-239 and Bommineni and Jauhar (1997) Maydica 42:107-120. Since the transformed material contains many cells; both transformed and non-transformed cells are present in any piece of subjected target callus or tissue or group of cells. The ability to kill non-transformed cells and allow transformed cells to proliferate results in transformed plant cultures. Often, the ability to remove non-transformed cells is a limitation to rapid recovery of transformed plant cells and successful generation of transgenic plants. Molecular and biochemical methods can be used to confirm the presence of the integrated heterologous gene of interest in the genome of transgenic plant.

Generation of transgenic plants may be performed by one of several methods, including, but not limited to, introduction of heterologous DNA by Agrobacterium into plant cells (Agrobacterium-mediated transformation), bombardment of plant cells with heterologous foreign DNA adhered to particles, and various other non-particle direct-mediated methods (e.g. Hiei et al. (1994) The Plant Journal 6:271-282; Ishida et al. (1996) Nature Biotechnology 14:745-750; Ayres and Park (1994) Critical Reviews in Plant Science 13:219-239; Bommineni and Jauhar (1997) Maydica 42:107-120) to transfer DNA.

Methods for transformation of chloroplasts are known in the art. See, for example, Svab et al. (1990) Proc. Natl. Acad. Sci. USA 87:8526-8530; Svab and Maliga (1993) Proc. Natl. Acad. Sci. USA 90:913-917; Svab and Maliga (1993) EMBO J. 12:601-606. The method relies on particle gun delivery of DNA containing a selectable marker and targeting of the DNA to the plastid genome through homologous recombination. Additionally, plastid transformation can be accomplished by transactivation of a silent plastid-borne transgene by tissue-preferred expression of a nuclear-encoded and plastid-directed RNA polymerase. Such a system has been reported in McBride et al. (1994) Proc. Natl. Acad. Sci. USA 91:7301-7305.

The cells that have been transformed may be grown into plants in accordance with conventional ways. See, for example, McCormick et al. (1986) Plant Cell Reports 5:81-84. These plants may then be grown, and either pollinated with the same transformed strain or different strains, and the resulting hybrid having constitutive expression of the desired phenotypic characteristic identified. Two or more generations may be grown to ensure that expression of the desired phenotypic characteristic is stably maintained and inherited and then seeds harvested to ensure expression of the desired phenotypic characteristic has been achieved. In this manner, the present invention provides transformed seed (also referred to as “transgenic seed”) having a nucleotide construct of the invention, for example, an expression cassette of the invention, stably incorporated into their genome.

E. Evaluation of Plant Transformation

Following introduction of heterologous foreign DNA into plant cells, the transformation or integration of the heterologous gene in the plant genome is confirmed by various methods such as analysis of nucleic acids, proteins and metabolites associated with the integrated gene.

PCR analysis is a rapid method to screen transformed cells, tissue or shoots for the presence of incorporated gene at the earlier stage before transplanting into the soil (Sambrook and Russell (2001) Molecular Cloning: A Laboratory Manual (Cold Spring Harbor Laboratory Press, Cold Spring Harbor, N.Y.)). PCR is carried out using oligonucleotide primers specific to the gene of interest or Agrobacterium vector background, etc.

Plant transformation may be confirmed by Southern blot analysis of genomic DNA (Sambrook and Russell (2001) supra). In general, total DNA is extracted from the transformant, digested with appropriate restriction enzymes, fractionated in an agarose gel and transferred to a nitrocellulose or nylon membrane. The membrane or “blot” can then be probed with, for example, radiolabeled ³²P target DNA fragment to confirm the integration of the introduced gene in the plant genome according to standard techniques (Sambrook and Russell, 2001, supra).

In Northern analysis, RNA is isolated from specific tissues of transformant, fractionated in a formaldehyde agarose gel, and blotted onto a nylon filter according to standard procedures that are routinely used in the art (Sambrook and Russell (2001) supra). Expression of RNA encoded by grg sequences of the invention is then tested by hybridizing the filter to a radioactive probe derived from a GDC by methods known in the art (Sambrook and Russell (2001) supra)

Western blot and biochemical assays and the like may be carried out on the transgenic plants to determine the presence of protein encoded by the herbicide resistance gene by standard procedures (Sambrook and Russell (2001) supra) using antibodies that bind to one or more epitopes present on the herbicide resistance protein.

F. Plants and Plant Parts

By “plant” is intended whole plants, plant organs (e.g., leaves, stems, roots, etc.), seeds, plant cells, propagules, embryos and progeny of the same. Plant cells can be differentiated or undifferentiated (e.g., callus, suspension culture cells, protoplasts, leaf cells, root cells, phloem cells, pollen). The present invention may be used for introduction of polynucleotides into any plant species, including, but not limited to, monocots and dicots. Examples of plants of interest include, but are not limited to, corn (maize), sorghum, wheat, sunflower, tomato, crucifers, peppers, potato, cotton, rice, soybean, sugarbeet, sugarcane, tobacco, barley, and oilseed rape, Brassica sp., alfalfa, rye, millet, safflower, peanuts, sweet potato, cassaya, coffee, coconut, pineapple, citrus trees, cocoa, tea, banana, avocado, fig, guava, mango, olive, papaya, cashew, macadamia, almond, oats, vegetables, ornamentals, and conifers.

Vegetables include, but are not limited to, tomatoes, lettuce, green beans, lima beans, peas, and members of the genus Curcumis such as cucumber, cantaloupe, and musk melon. Ornamentals include, but are not limited to, azalea, hydrangea, hibiscus, roses, tulips, daffodils, petunias, carnation, poinsettia, and chrysanthemum. Crop plants are also of interest, including, for example, maize, sorghum, wheat, sunflower, tomato, crucifers, peppers, potato, cotton, rice, soybean, sugarbeet, sugarcane, tobacco, barley, oilseed rape, etc.

This invention is suitable for any member of the monocot plant family including, but not limited to, maize, rice, barley, oats, wheat, sorghum, rye, sugarcane, pineapple, yams, onion, banana, coconut, and dates.

G. Methods for Increasing Plant Yield

Methods for increasing plant yield are provided. The methods comprise introducing into a plant or plant cell a polynucleotide comprising an EPSP synthase sequence having a sequence domain disclosed herein. As defined herein, the “yield” of the plant refers to the quality and/or quantity of biomass produced by the plant. By “biomass” is intended any measured plant product. An increase in biomass production is any improvement in the yield of the measured plant product. Increasing plant yield has several commercial applications. For example, increasing plant leaf biomass may increase the yield of leafy vegetables for human or animal consumption. Additionally, increasing leaf biomass can be used to increase production of plant-derived pharmaceutical or industrial products. An increase in yield can comprise any significant increase including, but not limited to, at least a 1% increase, at least a 3% increase, at least a 5% increase, at least a 10% increase, at least a 20% increase, at least a 30%, at least a 50%, at least a 70%, at least a 100% or a greater increase.

In specific methods, the plant is treated with an effective concentration of an herbicide, where the herbicide application results in enhanced plant yield. By “effective concentration” is intended the concentration which allows the increased yield in the plant. Such effective concentrations for herbicides of interest are generally known in the art. The herbicide may be applied either pre- or post emergence in accordance with usual techniques for herbicide application to fields comprising crops which have been rendered resistant to the herbicide by heterologous expression of an EPSP synthase gene of the invention.

Methods for conferring herbicide resistance in a plant or plant part are also provided. In such methods, an EPSP synthase polynucleotide disclosed herein is introduced into the plant, wherein expression of the polynucleotide results in glyphosate tolerance or resistance. Plants produced via this method can be treated with an effective concentration of an herbicide and display an increased tolerance to the herbicide. An “effective concentration” of an herbicide in this application is an amount sufficient to slow or stop the growth of plants or plant parts that are not naturally resistant or rendered resistant to the herbicide.

H. Methods of Controlling Weeds in a Field

Methods for selectively controlling weeds in a field containing a plant are also provided. In one embodiment, the plant seeds or plants are glyphosate resistant as a result of a polynucleotide having a sequence domain disclosed herein being inserted into the plant seed or plant. In specific methods, the plant is treated with an effective concentration of an herbicide, where the herbicide application results in a selective control of weeds or other untransformed plants. By “effective concentration” is intended the concentration which controls the growth or spread of weeds or other untransformed plants without significantly affecting the glyphosate-resistant plant or plant seed. Such effective concentrations for herbicides of interest are generally known in the art. The herbicide may be applied either pre- or post emergence in accordance with usual techniques for herbicide application to fields comprising plants or plant seeds which have been rendered resistant to the herbicide.

The following examples are offered by way of illustration and not by way of limitation.

### EXPERIMENTAL

### Example 1

### syngrg1 Design and Expression

A novel gene sequence encoding the GRG1 protein (SEQ ID NO:2; U.S. patent application Ser. No. 10/739,610) was designed and synthesized. This sequence is provided as SEQ ID NO:3. This open reading frame, designated “syngrg1” herein, was cloned into the expression vector pRSF1b (Invitrogen), by methods known in the art.

### Example 2

### Site Directed Mutagenesis of GRG1

U.S. patent application Ser. No. 11/651,752 filed Jan. 12, 2006 (herein incorporated by reference) discloses the Q-loop as an important region in conferring glyphosate resistance to EPSP synthases. The Q-loop is defined as the region from the valine corresponding to amino acid position 80 of SEQ ID NO:2 (GRG1) to the glutamine corresponding to amino acid position 105 of SEQ ID NO:2. For the purposes of the present invention, discussion of the Q-loop will be further restricted to a region comprising the “core” region of the Q-loop spanning from the isoleucine corresponding to amino acid position 84 of SEQ ID NO:2 to the isoleucine corresponding to amino acid position 99 of SEQ ID NO:2.

Herein a position number is assigned to the amino acids in this core region to simplify referral to each amino acid residue in this region. Thus, the positions of the Q-loop core correspond to amino acids 84 through 99 of SEQ ID NO:2 (I-D-C-G-E-S-G-L-S-I-R-M-F-T-P-I) and are herein designated as follows:

TABLE 1
Designation of Position Coordinates for Q-loop Core amino acids

| Amino Acid in | |
|---|---|
| GRG1 (SEQ ID | |
| NO: 2) | Designated Position in Q- |
| (single letter code) | loop Core |
| I | Position 1 |
| D | Position 2 |
| C | Position 3 |
| G | Position 4 |
| E | Position 5 |
| S | Position 6 |
| G | Position 7 |
| L | Position 8 |
| S | Position 9 |
| I | Position 10 |
| R | Position 11 |
| M | Position 12 |
| F | Position 13 |
| T | Position 14 |
| P | Position 15 |
| I | Position 16 |

To facilitate the mutagenesis of the syngrg1 gene, a variant of syngrg1 was generated that created convenient restriction sites flanking the Q-loop. This variant DNA sequence encodes a protein identical to the GRG1 protein. Mutagenesis of syngrg1 was performed using the QUIKCHANGE® Multisite kit (Stratagene, La Jolla, Calif.) using GATGGCAGCCTCCAGATCACTAGTGAAGGCGTTAAGCCAGTGGC (SEQ ID NO:52) and GTTCACACCAATCGTGGCGCTTTCGAAGGAAGAAGTGACAATCAAG (SEQ ID NO:53) oligonucleotides to simultaneously introduce two restriction sites flanking the Q-loop region of GRG1; an Spe I site 5′ of the loop, and a BstB I site 3′ to the Q-loop region. The DNA sequence of the resulting clone, ‘syngrg1-SB’ (SEQ ID NO:4) was confirmed by DNA sequencing.

### Example 3

### Combinatorial Mutagenesis of syngrg1-SB

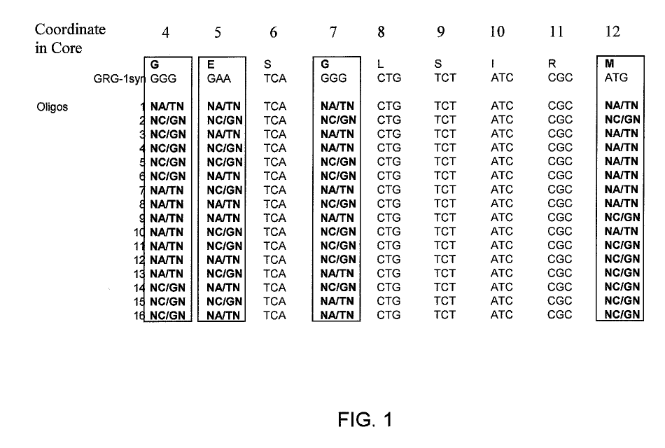

A library of mutant clones (Library1) was developed by combinatorial mutagenesis within the Q-loop core region of GRG1 with a set of 32 oligonucleotides. These oligonucleotides were designed to introduce mutations in four of the Q-loop residues, at positions 4, 5, 7, and 12 of the Q-loop Core (Table 1 and FIG. 1). Oligonucleotides were resuspended in 10 mM Tris-HCl pH 8.5 at a concentration of 10 μM. To form double-stranded DNA molecules, complementary oligonucleotides were mixed and incubated as follows: 95° C. for 1 minute; 80° C. for 1 minute; 70° C. for 1 minute; 60° C. for 1 minute; and 50° C. for 1 minute.

The double-stranded DNA molecules containing degenerate codons were digested with Spe I and BstB I restriction enzymes as specified by the manufacturer. After the restriction digest, the DNA was loaded onto a 4% agarose gel and subjected to electrophoresis. The DNA was excised from the gel and eluted using a QIAQUICK® gel extraction kit (Qiagen, Valencia, Calif.). The annealed oligonucleotides were ligated into pRSF1b-syngrg-SB, digested with Spe I and BstB I, treated with calf alkaline phosphatase, transformed into BL21*DE3 cells (Invitrogen), and plated onto LB plates containing kanamycin. From these test transformations, the library was estimated to contain approximately 140,000 clones. To confirm the diversity of the library, 20 clones were randomly picked from the LB-kanamycin plates and sequenced in the Q-loop region. This sequence analysis confirmed that the high diversity of the library, and demonstrated that 75% of the clones were full length and possessed and intact open reading frame in the Q-loop region (data not shown).

### Example 4

### Permutational Mutagenesis of syngrg1-SB

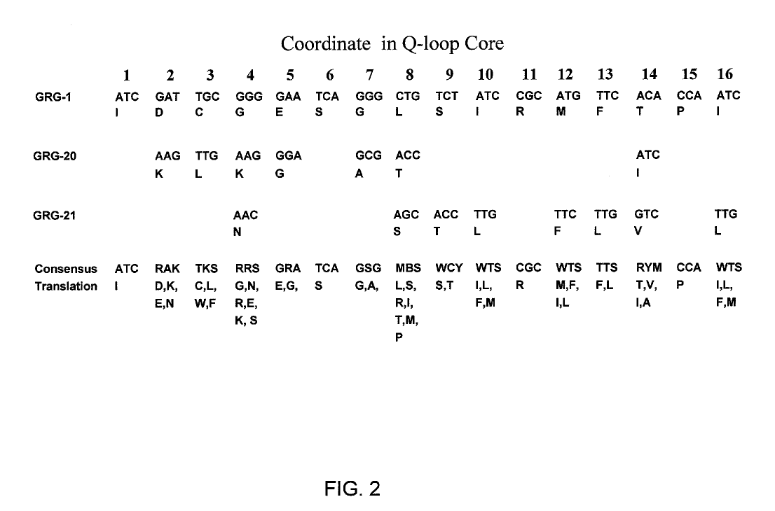

The method of permutational mutagenesis (U.S. Patent Application No. 60/813,095, filed Jun. 13, 2006 and incorporated herein by reference in its entirety) was used to generate a second library of variants in the Q-loop. The amino acid sequences of GRG1, GRG20 (SEQ ID NO:54; U.S. Patent Application No. 60/658,320) and GRG21 (SEQ ID NO:55; U.S. Patent Application No. 60/658,320) were aligned, and a consensus set of amino acids developed (FIG. 2).

A series of oligonucleotides was designed to introduce the diversity represented in FIG. 2, which covers the full diversity of the consensus translation of the Q-loop core as shown in Table 3. Positions 1, 6, 11, and 15 are absolutely conserved between GRG1, GRG20, and GRG21. The potential diversity generated by this approach is shown as the consensus translation in FIG. 2 and in SEQ ID NO:48.

Oligonucleotides were resuspended in 10 mM Tris-HCl pH 8.5 at a concentration of 10 uM. To form double stranded DNA molecules, complementary oligonucleotides were mixed and incubated as follows: 95° C. for 1 minute; 80° C. for 1 minute; 70° C. for 1 minute; 60° C. for 1 minute; and 50° C. for 1 minute. The annealed oligonucleotides were ligated to pRSF1b-syngrg1-SB digested with Spe I and BstB I, and treated with calf alkaline phosphatase. Test ligations were transformed into BL21*DE3 (Invitrogen) and plated on LB-kanamycin. From these test transformations, the library was estimated to contain approximately 180,000 clones. Twenty clones were randomly selected from the clones growing on LB and sequenced. Nineteen of the 20 clones were found to encode full length, in-frame proteins in the Q-loop region, despite the generation of a large amount of diversity in the region. High degrees of variation were seen (at all 13 target positions) in the twenty clones sequenced, suggesting that the library diversity approached its theoretical level (data not shown).

### Example 5

### Screening for Glyphosate Resistance on Plates

Library ligations were transformed into BL21*DE3 competent E. coli cells (Invitrogen). The transformations were performed according to the manufacturer's instructions with the following modifications. After incubation for 1 hour at 37° C. in SOC medium, the cells were sedimented by centrifugation (5 minutes, 1000×g, 4° C.). The cells were washed with 1 ml M63+, centrifuged again, and the supernatant decanted. The cells were washed a second time with 1 ml M63+ and resuspended in 200 ul M63+.

For selection of mutant GRG1 enzymes conferring glyphosate resistance in E. coli, the cells were plated onto M63+ agar medium plates containing 50 mM glyphosate, 0.05 mM IPTG (isopropyl-beta-D-thiogalactopyranoside), and 50 ug/ml kanamycin. M63+ medium contains 100 mM KH₂PO₄, 15 mM (NH₄)₂SO₄, 50 μM CaCl₂, 1 μM FeSO₄, 50 μM MgCl₂, 55 mM glucose, 25 mg/liter L-proline, 10 mg/liter thiamine HCl, sufficient NaOH to adjust the pH to 7.0, and 15 g/liter agar. The plates were incubated for 36 hours at 37° C.

### Example 6

### Preparation of Extracts Containing Glyphosate Resistant GRG-1 Mutants

BL21*DE3 cells transformed with GRG-1 mutants growing on glyphosate plates were grown in LB medium supplemented with 50 ug/ml kanamycin at 37° C. When the culture media reached an optical density (600 nm) of 0.5, 0.5 mM IPTG was added, and the cultures were incubated for 16 hours at 20° C. The cultures were centrifuged at 12,000×g for 15 minutes at 4° C., the supernatant was removed, and the cells were resuspended in 50 mM Hepes/KOH pH 7.0, 300 mM NaCl, 1 mg/ml lysozyme, 0.04 ml DNase I. The resuspended cells were incubated for 1 hour at room temperature. The cells were sonicated 3 times for 10 seconds using a Misonix Sonicator 3000 at setting 7.5. Between sonication bursts the cells were incubated on ice for 30 seconds. The cell lysates were centrifuged at 27000×g for 15 minutes at 4° C., and the supernatant comprising the cell extract was recovered. The cell extracts were dialyzed 2× for 4 hours against 50 mM Hepes/KOH pH 7.0, 300 mM NaCl and stored at 4° C.

### Example 7

### Quantification of Extracts Containing Glyphosate Resistant GRG-1 Mutants

The expression of GRG-1 variant proteins in cell extracts was determined by a quantitative antibody dot blot. Two sheets of 3 mM filter paper were soaked in 1×PBS buffer (20 mM potassium phosphate pH 7.2, 150 mM NaCl) and placed in a 96 well dot blot manifold (Schleicher and Schuell, Keene, N.H.). One sheet of Optitran BA-S 83 cellulosenitrate membrane (Schleicher and Schuell) was soaked in 1×PBS buffer and placed on top of the 3 mM filter paper. Serial dilutions of cell extracts as well as dilutions of purified GRG-1 wild-type protein of known concentration (“protein standards”) were prepared in a final volume of 100 ul 1×PBS. The samples were loaded into the dot blot wells and a vacuum of 10 cm Hg was applied. The wells were washed 3× times with 300 ul PBS. The cellulosenitrate membrane was removed and blocked for one hour in 3% dry milk in PBS. The blocking solution was removed and the cellulosenitrate membrane was incubated with an anti-6×His monoclonal antibody conjugated to horseradish peroxidase (Serotec, Raleigh, N.C.) diluted 1:5000 in 3% dry milk in PBS. After one hour incubation at room temperature, the membrane was washed four times for five minutes with PBS-T (0.05% Tween20 in PBS). The membrane was incubated with ECL PLUS™ western blotting detection reagent (Amersham Biosciences, Piscataway, N.J.) for five minutes at room temperature. The detection solution was removed and a Biomax Light film (Kodak) was placed on top of the membrane and exposed for ten minutes. The film was scanned and signal quantitation was performed using Phoretix Array software (Nonlinear Dynamics, Durham, N.C.) by comparison to the GRG1 protein standards.

### Example 8

### Determination of EPSPS Activity of GRG-1 Variants

Extracts containing GRG1 variant proteins were assayed for EPSP synthase activity using assays as previously described (U.S. Patent Application No. 60/741,166, herein incorporated by reference in its entirety). Assays were typically carried out in a final volume of 50 ul containing 0.5 mM shikimate-3-phosphate, 0-500 uM phosphoenolpyruvate (PEP), 1 U/ml xanthine oxidase, 2 U/ml nucleoside phosphorilase, 2.25 mM inosine, 1 U/ml horseradish peroxidase, 0-2 mM glyphosate, 50 mM Hepes/KOH pH 7.0, 100 mM KCl, and AMPLEX® Red (Invitrogen) according to the manufacturer's instructions. Extracts were typically incubated with all assay components except shikimate-3-phosphate and AMPLEX™ Red for 5 minutes at room temperature, and assays were started by adding shikimate-3-phosphate and AMPLEX® Red. EPSP synthase activity was measured using a Spectramax Gemini XPS fluorescence spectrometer (Molecular Dynamics, excitation: 555 nm; emission: 590 nm).

For initial determination of kinetic parameters, assays were performed at a single PEP concentration of 50 uM, and the activity of the enzymes assessed at 0.1 mM, and 2 mM glyphosate. Clones whose extracts showed little or no difference in activity at 2 mM vs. 1 mM glyphosate were selected for full kinetic analysis.

Following full determination of kinetic parameters, the kinetic constants were determined as follows, adjusting for the quantity of protein determined by either antibody dot-blot analysis as described herein, or by Bradford Assay, as known in the art. For any one glyphosate concentration, EPSP synthase activity was measured as a function of a broad range of PEP concentrations. The data were fit to the Michaelis-Menten equation using KALEIDAGRAPH® software (Synergy Software) and used to determine the Kₘ (Kₘ apparent) of the EPSP synthase at that glyphosate concentration. Kₘ apparent values were determined at no fewer than 3 glyphosate concentrations, and the Kᵢ of the EPSPS for glyphosate were calculated from the plot of Kₘ apparent vs. glyphosate concentration, using the equation (ml*x/(m2+x); ml=1; m2=1) as known in the art.

### Example 9

### Determination of Variant Residues from Library 1

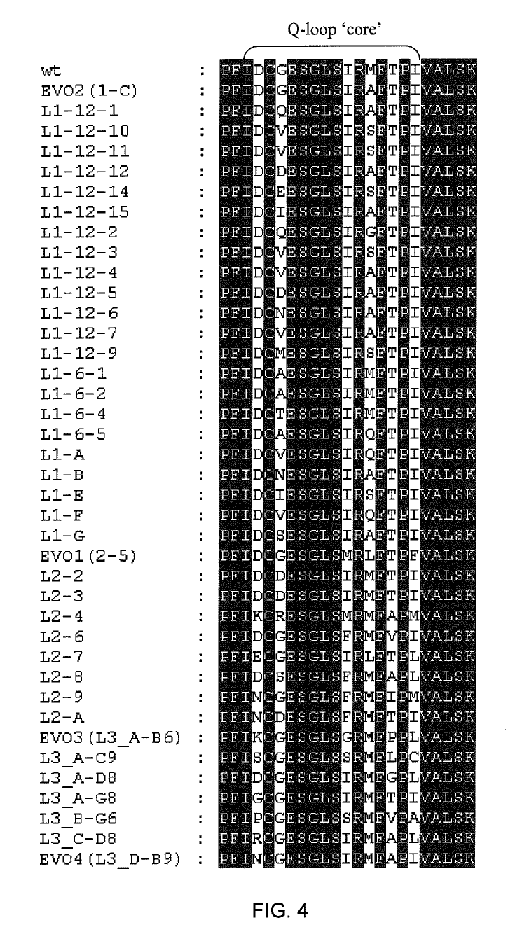

For Library 1, 23 clones were identified by growth on 50 mM glyphosate plates. DNA was isolated from these twenty three clones, and the DNA sequence of the Q-loop regions of the clones was determined. Kinetic analysis was performed on crude extracts of all 23 clones (FIG. 4 and SEQ ID NO:5-43). Of the clones identified from Library 1, clone L1-C was determined to have the highest level of glyphosate resistance, and full kinetic analysis was performed. This clone is herein designated syngrg1(evo2) and the encoded protein designated GRG1(EVO1) (SEQ ID NO:5).

Comparison of the resulting DNA sequences against the DNA sequences of the randomly sampled clones revealed that, of the four core residues altered in Library 1, two of the four were intolerant of variation. For example, for position 5 of the core region, only clones encoding glutamic acid were represented in Library 1 under the conditions used in this assay (e.g., growth on 50 mM glyphosate). This suggests that substitution of the other amino acids for glutamic acid at this position did not result in a detectable glyphosate-resistant clone. From this analysis it is determined that, for position 5, glutamic acid is the preferred residue and, for position 7, glycine is the preferred amino acid for enhancement of glyphosate resistance under the conditions of the disclosed assay. It is possible that additional resistant clones representing further diversity in this region could be identified from a screening at lower concentrations of glyphosate (e.g., 20 mM, 15 mM, 10 mM, or lower) or after longer incubation times (e.g., greater than 16 hours). In fact, Example 12, infra, demonstrates that, as the concentration of glyphosate increases, the number of resistant clones decreases.

TABLE 1
Mutagenesis of Library 1

| | Amino | | | |
|---|---|---|---|---|
| | Acid | | | AA |
| Position | (AA) | AAs represented in | AAs identified in | present |
| of Core | in GRG1 | Library | resistant clones | in EVO2 |
| 4 | G | All 20 amino acids | G, Q, V, D, E, I, | G |
| | | | N, M, A, T, S | |
| 5 | E | All 20 amino acids | E | E |
| 7 | G | All 20 amino acids | G | G |
| 12 | M | All 20 amino acids | M, A, S, G, Q | A |

### Example 10

### Determination of Variant Residues from Library 2

Library 2 has a theoretic diversity of over 2,000,000 clones, and approximately 180,000 clones were tested for glyphosate resistance. Nine clones were identified by growth on 50 mM glyphosate plates. DNA was isolated from these nine clones, and the DNA sequence of the Q-loop regions of the clones was determined. Comparison of the resulting DNA sequences against the DNA sequences of the randomly sampled clones showed that many of the 13 core residues altered in Library 2 were intolerant of variation (see Table 3). For example, position 8 of the core region was represented by the amino acids leucine, isoleucine, serine, arginine, methionine, and proline in Library 2. However, every glyphosate resistant clone (growing on 50 mM glyphosate) isolated contained a Leucine at this Position 8. Thus, this method is useful to “map” the mutable amino acids in the core region.

Of the clones identified from Library 2, clone 2-5 was determined based on kinetic analysis to have the highest glyphosate resistance under the conditions of this assay, and is herein designated syngrg1 (evo1) and the encoded protein designated GRG1(EVO1) (SEQ ID NO:28).

TABLE 3
Mutagenesis of Library 2.

| | Amino | | | |
|---|---|---|---|---|
| | Acid | | | AA |
| Position | (AA) | AAs represented in | AAs identified in | present |
| of Core | in GRG1 | Library | resistant clones | in EVO2 |
| 2 | D | D, K, E, N | D, K, E | D |
| 3 | C | C, L, W, F | C | C |
| 4 | G | G, N, R, E, K, S, D | G, D, R, G, S | G |
| 5 | E | E, G | E | E |
| 7 | G | G, A | G | G |
| 8 | L | L, S, R, I, T, M, P | L | L |
| 9 | S | S, T | S | S |
| 10 | I | I, L, F, M | I, M, F | M |
| 12 | M | M, F, I, L | M, L | L |
| 13 | F | F, L | F | F |
| 14 | T | T, V, I, A | T, A, V, I | T |
| 16 | I | I, L, F, M | I, L, F, M | F |

### Example 11

### Generation and Isolation of evo3 and evo4

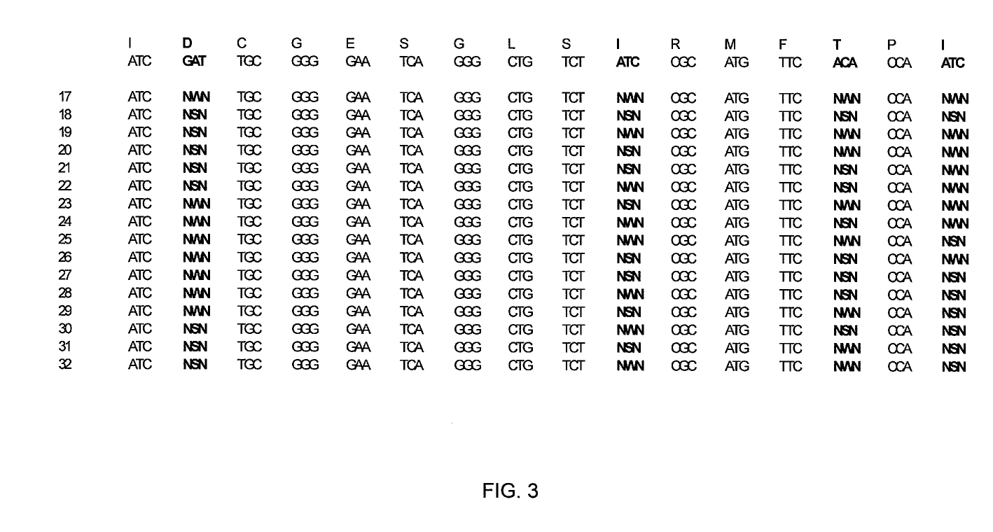

A third library was generated using the methods above, capitalizing on the information from Libraries 1 and 2 such that only residues known to be mutable were utilized. Library 3 was generated using populations of oligos that encode every amino acid possibility at positions 2, 10, 14, and 16 (see FIG. 3). Library 3 was generated as described above for Library 1, transformed into E. coli, and tested for clones conferring glyphosate resistance in a manner similar to that for Libraries 1 and 2. Approximately 150,000 libraries were screened by growth on M63+ plates with 50 mM glyphosate, kanamycin and IPTG as above. Two hundred and ninety two clones were identified as growing on these 50 mM glyphosate. These two hundred and ninety two clones were then re-struck onto similar plates containing either 100 mM or 200 mM glyphosate. Seven clones were identified as growing on 200 mM glyphosate, and all seven were directly analyzed for their kinetic properties. The DNA sequence of the Q-loop region of these clones was then determined. Two clones, syngrg1(evo3) (SEQ ID NO:37; also referred to herein as evo3), and syngrg1(evo4) (SEQ ID NO:43; also referred to herein as evo4), were identified as having the largest improvement in kinetic parameters.

TABLE 4
Q-loop variant sampling in Library 3.

| | Amino | | | | |
|---|---|---|---|---|---|
| Posi- | Acid | | | | |
| tion | (AA) | AAs repre- | | AA | AA |
| of | in | sented in | AAs identified in | present | present |
| Core | GRG1 | Library | resistant clones | in EVO3 | in EVO4 |
| 2 | D | All | D, K, S, G, P, R, | K | N |
| | | | N | | |
| 10 | I | All | I, G, S | G | I |
| 14 | T | All | T, P, L, G, V, A | P | A |
| 16 | I | All | I, L, C, A | L | I |

### Example 12

### EVO1 EVO2, EVO3, and EVO4 have Improved Kinetic Properties

Kinetic analysis of EVO1, EVO2, EVO3, and EVO4 demonstrates that all four of these proteins exhibit improved glyphosate resistance relative to GRG1 (Table 5). All four proteins exhibit an improved Kᵢ for glyphosate and retain a reasonable Kₘ for PEP below 200 mM. EVO3 exhibits very high glyphosate resistance, and a Kₘ for PEP that is virtually identical to GRG1. EVO4 has very high glyphosate resistance, and a reasonable, though somewhat elevated Kₘ for PEP.

TABLE 5
Kinetics of EVO1-EVO4 vs. GRG1.

| | GRG-1 | | | | |
|---|---|---|---|---|---|
| | wt | EVO1 | EVO2 | EVO3 | EVO4 |
| Ki (μM) vs. GRG1 | ++ | +++ | +++ | ++++ | ++++ |

### Example 13

### Additional Combinations of Q-Loop Variants; Library 4

Given the identification of functional residues in the Q-loop core, and given the identification of clones with improved function, one can generate an additional library that combines the altered residues identified in the improved clones. One method to achieve this library is to generate a combinatorial library using oligonucleotides, in a manner similar to Library 1. Alternatively, one may generate a permutational library as in Library 2.

The amino acid sequences of EVO1, EVO2, EVO3 and EVO4 were aligned, consensus translation was derived (SEQ ID NO:50), and oligonucleotides designed to generate a permutational library. This library has a theoretical diversity of approximately 1500 clones. The oligonucleotides were annealed as described herein, and ligated to pRSF1b-syngrg1-SB which was digested with Spe I and BstB I, and treated with calf alkaline phosphatase. The resulting library was plated on M63+ plates (as described above) containing 50 mM glyphosate. Fourteen clones were identified as growing on 50 mM glyphosate.

Protein expressed from these fourteen clones was tested for (1) improved resistance to glyphosate and (2) unperturbed affinity for PEP. Six of these fourteen clones [grg1(4S-10) (SEQ ID NO:66), grg1(4S-16) (SEQ ID NO:69); grg1(4S-28) (SEQ ID NO:70); grg1(4S-3) (SEQ ID NO:71); grg1(4S-39) (SEQ ID NO:72); and grg1(4S-60) (SEQ ID NO:73)] encoding proteins GRG1(4S-10) (SEQ ID NO:56); GRG1(4S-16) (SEQ ID NO:59); GRG1(4S-28) (SEQ ID NO:60); GRG1(4S-3) (SEQ ID NO:61); GRG1(4S-39) (SEQ ID NO:62); and GRG1(4S-60) (SEQ ID NO:63), respectively, demonstrated improved glyphosate resistance and good affinity for PEP. grg1 (4S-10) was renamed grg1 (evo5) (SEQ ID NO:66), and the protein it encodes was designated as GRG1(EVO5) (SEQ ID NO:56). Kinetic analysis of GRG1(EVO5) protein (Table 6), determined that GRG1(EVO5) has a kᵢ/kₘ ratio of 1769.

TABLE 6
Kinetics of selected variants.

| Variant | kₘ (μM) | kᵢ (μM) | ki/kₘ |
|---|---|---|---|
| wt | 22 | 152 | 7 |
| 4s-3 | 26 | 1,375 | 53 |
| 4s-10 | 5 | 8,757 | 1,769 |
| [GRG1(EVO5)] | | | |
| 4s-16 | 23 | nd | nd |
| 4s-28 | 4.5 | 606 | 134 |
| 4s-39 | 2.8 | 851 | 304 |

The amino acid changes identified in these six variants provide further delineation of the key residues in the Q-loop contributing to glyphosate resistance.

TABLE 7
Q-loop variant sampling in Library 5.

| | Amino | | | |
|---|---|---|---|---|
| | Acid | | | AA |
| Position | (AA) | AAs represented in | AAs identified in | present |
| of Core | in GRG1 | Library | resistant clones | in EVO5 |
| 2 | D | N, K, E, D | K, E | E |
| 10 | I | S, I, M, G, V, R | S, G, V | S |
| 12 | M | T, I, M, A, V, S, L | I, M, A, L | I |
| 14 | T | T, P, A | T, P | T |
| 16 | I | I, L, F | I, L, F | L |

### Example 14

### Random Mutagenesis to Increase Solubility of GRG1(EVO5); Library 5

DNA from grg1 (evo5) was mutagenized by PCR as known in the art, and cloned into pRSF1b (Invitrogen), and a library of mutagenized clones identified by virtue of growth on 50 mM glyphosate. These clones were then analyzed by quantitative dot blot as described herein, and two clones with modifications relative to grg1 (evo5) [(grg1(5.2.A10) (SEQ ID NO:67) and grg1(5.2.B6) (SEQ ID NO:68)], encoding GRG1(5.2.A10) (SEQ ID NO:57) and GRG1(5.2.B6) (SEQ ID NO:58), respectively, were selected by virtue of demonstrating a substantial increase in soluble protein. GRG1(5.2.A10) (SEQ ID NO:57) and GRG1(5.2.B6) (SEQ ID NO:58) each have a single amino acid change relative to GRG1(EVO5). These changes are shown in Table 8. The EPSPS coding region of 5.2.A10 was renamed as grg1 (evo6) (SEQ ID NO:67), and its encoded protein as GRG1(EVO6) (SEQ ID NO:57).

TABLE 8
Summary of mutations in Variants
5.2.A10 (GRG1(EVO6)) and 5.2.B6

| | Amino acid change relative to |
|---|---|
| Variant | GRG1(EVO5) |
| 5.2.A10 [GRG1(EVO6)] | N186 →K |
| 5.2.B6 | V351 →A |

### Example 15

### grg1(evo7) and grg1 (evo8)

grg1(evo7) (SEQ ID NO:74), encoding the GRG1(EVO7) protein (SEQ ID NO:64), and grg1(evo8) (SEQ ID NO:75), encoding the GRG1(EVO8) protein (SEQ ID NO:65), were isolated as glyphosate resistant clones after mutagenesis of grg1 (evo6) in the Q-loop region. Kinetic analysis of GRG1(EVO7) and GRG1(EVO8) show that both clones have further improved kinetic properties over GRG1(EVO6).

TABLE 9
Kinetics of selected variants

| | GRG1(EVO6) | GRG1(EVO7) | GRG1(EVO8) |
|---|---|---|---|
| kᵢ (μM) | 7,211 | 16,200 | 1,426 |
| kₘ (μM) | 16 | 13 | 20 |
| Vₘₐₓ | 7 | 15 | 6 |
| (nmol/min/ug) | | | |
| kᵢ/kₘ | 451 | 1,246 | 71 |

### Example 16

### Summary of Mutagenesis of Q-Loop Core Region

Given the extensive mutagenesis of the Q-loop core region, the isolation of variants that continue to exhibit glyphosate resistance, and the isolation of improved variants, one can infer the key amino acids responsible for conferring improved glyphosate resistance in a GRG1 backbone. The data is summarized in Table 10.

TABLE 10
Summary of mutagenesis of GRG1

| | | Amino | | AAs | | | | | | | | |
|---|---|---|---|---|---|---|---|---|---|---|---|---|
| Position | Library | Acid | AAs | Identified | | | | | | | | |
| of | Where | (AA) | Represented | in Resistant | AA CHANGES in EVO1, 2, 3, 4, 5, 6, 7, 8 | | | | | | | |
| Core | Tested | in GRG1 | in Library | Clones | 1 | 2 | 3 | 4 | 5 | 6 | 7 | 8 |
| 2 | L2, L3, L4 | D | All | D, K, E, S, | | | K | N | K | K | K | K |
| | | | | G, P, R, N | | | | | | | | |
| 3 | L2 | C | C, L, W, F | C | | | | | | | | |
| 4 | L1 ,L2 | G | All | G, Q, V, D, | | G | | | | | | |
| | | | | E, I, N, M, | | | | | | | | |
| | | | | A, T, S, R | | | | | | | | |
| 5 | L1, L2 | E | All | E | | | | | | | | |
| 7 | L1, L2 | G | All | G | | | | | | | | |
| 8 | L2 | L | L, S, R, I, T, | L | | | | | | | | |
| | | | M, P | | | | | | | | | |
| 9 | L2 | S | S, T | S | | | | | | | | |
| 10 | L2, L3, | I | All | I, G, S, M, | M | | G | I | S | S | S | S |
| | L4 | | | F, V | | | | | | | | |
| 12 | L1, L2, | M | All | M, A, S, G, | L | A | | | I | I | L | V |
| | L4 | | | Q, L, I | | | | | | | | |
| 13 | L2 | F | F, L | F | | | | | | | | |
| 14 | L2, L3, | T | All | T, P, L, G, | | | P | A | T | T | T | T |
| | L4 | | | A, V, I | | | | | | | | |
| 16 | L2, L3, | I | All | I, L, C, A, | F | | L | I | L | L | L | L |
| | L4 | | | F, M | | | | | | | | |

### Example 17

### Further Delineation of Key Amino Acids in Q-Loop

The amino acid sequence of the variants isolated here further expands and delineates the key amino acids tolerated and desired in this region. Thus, the variations of the Q-loop core domain (positions 2-16, corresponding to positions 85-99 of SEQ ID NO:2) that confer glyphosate resistance can be summarized by the expression X-C-X-E-S-G-L-S-X-R-X-F-X-P-X (SEQ ID NO:44), (where X is any amino n one embodiment, the domain is represented by X₁-C-X₂-E-S-G-L-S-X₃-R-X₄-X₆ (SEQ ID NO:45), and wherein X₁ denotes D, K, E, S, G, P, or R, or N, and X₂ G, Q, V, D, E, I, N, M, A, T, S, or R, and X₃ denotes I, G, S, M, F or V, X₄ denotes M, A, S, G, Q, L, V, or I, X₅ denotes T, P, L, G, A, V, or I, and X₆ denotes I, L, C, A, F, or M.

In another embodiment, the domain is represented by D-C-X₁-X₂-S-G (SEQ ID NO:76), wherein X₁ denotes glutamine, valine, proline, glutamic acid, isoleucine, methionine, or threonine and X₂ denotes any amino acid.

### Example 18

### Cloning of syngrg1, evo1, evo2, evo3, evo4, evo5, evo6, evo7, and evo8 into a Plant Expression Cassette

For each of syngrg1, evo1, evo2, evo3, evo4, evo5, evo6, evo7, and evo8, the open reading frame (ORF) is amplified by PCR from a full-length DNA template. Hind III restriction sites are added to each end of the ORFs during PCR. Additionally, the nucleotide sequence ACC is added immediately 5′ to the start codon of the gene to increase translational efficiency (Kozak (1987) Nucleic Acids Research 15:8125-8148; Joshi (1987) Nucleic Acids Research 15:6643-6653). The PCR product is cloned and sequenced using techniques well known in the art to ensure that no mutations are introduced during PCR.

The plasmid containing the PCR product is digested with Hind III and the fragment containing the intact ORF is isolated. This fragment is cloned into the Hind III site of a plasmid such as pAX200, a plant expression vector containing the rice actin promoter (McElroy et al. (1991) Molec. Gen. Genet. 231:150-160) and the PinII terminator (An et al. (1989) The Plant Cell 1:115-122). The promoter-gene-terminator fragment from this intermediate plasmid is then subcloned into plasmid pSB11 (Japan Tobacco, Inc.) to form a final pSB11-based plasmid. In some cases, it may be preferable to generate an alternate construct in which a chloroplast leader sequence is encoded as a fusion to the N-terminus of the syngrg1, evo1, evo2, evo3, evo4, evo5, evo6, evo7, and evo8 constructs. These pSB11-based plasmids are typically organized such that the DNA fragment containing the promoter-gene-terminator construct, or promoter-chloroplast leader-gene-terminator construct may be excised by double digestion by restriction enzymes, such as Kpn I and Pme I, and used for transformation into plants by aerosol beam injection. The structure of the resulting pSB11-based clones is verified by restriction digest and gel electrophoresis, and by sequencing across the various cloning junctions.

The plasmid is mobilized into Agrobacterium tumefaciens strain LBA4404 which also harbors the plasmid pSB1 (Japan Tobacco, Inc.), using triparental mating procedures well known in the art, and plating on media containing spectinomycin. The pSB11-based plasmid clone carries spectinomycin resistance but is a narrow host range plasmid and cannot replicate in Agrobacterium. Spectinomycin resistant colonies arise when pSB11-based plasmids integrate into the broad host range plasmid pSB1 through homologous recombination. The cointegrate product of pSB1 and the pSB11-based plasmid is verified by Southern hybridization. The Agrobacterium strain harboring the cointegrate is used to transform maize by methods known in the art, such as, for example, the PureIntro method (Japan Tobacco).

### Example 19

### Transformation of Maize Cells by Aerosol Beam

Maize ears are best collected 8-12 days after pollination. Embryos are isolated from the ears, and those embryos 0.8-1.5 mm in size are preferred for use in transformation. Embryos are plated scutellum side-up on a suitable incubation media, such as DN62A5S media (3.98 g/L N6 Salts; 1 ml/L (of 1000× Stock) N6 Vitamins; 800 mg/L L-Asparagine; 100 mg/L Myo-inositol; 1.4 g/L L-Proline; 100 mg/L Casamino acids; 50 g/L sucrose; 1 ml/L (of 1 mg/ml stock) 2.4-D). However, media and salts other than DN62A5S are suitable and are known in the art. Embryos are incubated overnight at 25° C. in the dark. However, it is not necessary per se to incubate the embryos overnight.

The resulting explants are transferred to mesh squares (30-40 per plate), transferred onto osmotic media for about 30-45 minutes, then transferred to a beaming plate (see, for example, PCT Publication No. WO/0138514 and U.S. Pat. No. 5,240,842).

DNA constructs designed to express the GRG proteins of the present invention in plant cells are accelerated into plant tissue using an aerosol beam accelerator, using conditions essentially as described in PCT Publication No. WO/0138514. After beaming, embryos are incubated for about 30 min on osmotic media, and placed onto incubation media overnight at 25° C. in the dark. To avoid unduly damaging beamed explants, they are incubated for at least 24 hours prior to transfer to recovery media. Embryos are then spread onto recovery period media, for about 5 days, 25° C. in the dark, then transferred to a selection media. Explants are incubated in selection media for up to eight weeks, depending on the nature and characteristics of the particular selection utilized. After the selection period, the resulting callus is transferred to embryo maturation media, until the formation of mature somatic embryos is observed. The resulting mature somatic embryos are then placed under low light, and the process of regeneration is initiated by methods known in the art. The resulting shoots are allowed to root on rooting media, and the resulting plants are transferred to nursery pots and propagated as transgenic plants.

### Materials

| Components | per liter | | Source |
|---|---|---|---|
| Chu's N6 Basal Salt | 3.98 | g/L | Phytotechnology Labs |
| Mixture (Prod. No. C 416) | | | |
| Chu's N6 Vitamin Solution | 1 ml/L (of | | Phytotechnology Labs |
| (Prod. No. C 149) | 1000× Stock) | | |
| L-Asparagine | 800 | mg/L | Phytotechnology Labs |
| Myo-inositol | 100 | mg/L | Sigma |
| L-Proline | 1.4 | g/L | Phytotechnology Labs |
| Casamino acids | 100 | mg/L | Fisher Scientific |
| Sucrose | 50 | g/L | Phytotechnology Labs |

| | | |
|---|---|---|
| 2,4-D (Prod. No. D-7299) | 1 ml/L (of | Sigma |
| | 1 mg/ml Stock) | |

Adjust the pH of the solution to pH 5.8 with 1N KOH/1N KCl, add Gelrite (Sigma) to 3 g/L, and autoclave. After cooling to 50° C., add 2 ml/L of a 5 mg/ml stock solution of Silver Nitrate (Phytotechnology Labs). Recipe yields about 20 plates.

### Example 20

### Transformation of EPSP Synthase Enzymes into Maize Plant Cells by Agrobacterium-Mediated Transformation

Ears are best collected 8-12 days after pollination. Embryos are isolated from the ears, and those embryos 0.8-1.5 mm in size are preferred for use in transformation. Embryos are plated scutellum side-up on a suitable incubation media, and incubated overnight at 25° C. in the dark.

However, it is not necessary per se to incubate the embryos overnight. Embryos are contacted with an Agrobacterium strain containing the appropriate vectors having an EPSP synthase enzyme with a Q-loop region domain of the present invention for Ti plasmid mediated transfer for about 5-10 min, and then plated onto co-cultivation media for about 3 days (25° C. in the dark). After co-cultivation, explants are transferred to recovery period media for about five days (at 25° C. in the dark). Explants are incubated in selection media for up to eight weeks, depending on the nature and characteristics of the particular selection utilized. After the selection period, the resulting callus is transferred to embryo maturation media, until the formation of mature somatic embryos is observed. The resulting mature somatic embryos are then placed under low light, and the process of regeneration is initiated as known in the art. The resulting shoots are allowed to root on rooting media, and the resulting plants are transferred to nursery pots and propagated as transgenic plants.

### Example 21

### Transgenic Plants Expressing grg1 (evo5) and grg1 (evo6)

The grg1 (evo5) gene and grg1 (evo6) genes were each cloned into a plant expression vector suitable for Agrobacterium-mediated transformation, such vector containing at least (1) a promoter capable of expression in a plant cell, (2) a chloroplast peptide leader coding sequence, (3) a transcriptional terminator. The resulting clones, pAX4014 and pAX4032 respectively, were transferred to Agrobacterium as known in the art and described herein, and the resulting Agrobacterium strain used to develop transgenic maize callus and ultimately transgenic maize plants.

Western blot analysis of transgenic plants showed expression of GRG1 (EVO5) in the grg1 (evo5) transgenic plant tissue, and expression of GRG1 (EVO6) in the grg1 (evo6) transgenic plant tissue. Tₒ transgenic plants were sprayed with glyphosate formulations of 14 mM glyphosate after adaptation to soil, and the resistance to the herbicide was scored after two weeks relative to non-transgenic controls.

TABLE 11
Glypohosate Resistant Plants

| | | | Survival | | | |
|---|---|---|---|---|---|---|
| | | Expres- | at 2 | | | |
| | Number | sion of | weeks | | | |
| | of | EPSPS | post- | Chloro- | Stunt- | Seed |
| EPSPS Gene | Events | observed | spray? | sis? | ing? | Set? |
| GRG1 (EVO5) | >10 | YES | YES | NONE | NONE | YES |
| GRG1(EVO6) | >10 | YES | YES | NONE | NONE | YES |
| Non- | N/A | NO | NO | N/A | N/A | N/A |
| transformed | | | | | | |
| control plants | | | | | | |

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claims.

That which is claimed:

1. An isolated polynucleotide other than SEQ ID NO: 1 and 46 encoding a glyphosate tolerance EPSP synthase polypeptide having a sequence domain selected from the group consisting of:
a) X-C-X-E-S-G-L-S-X-R-X-F-X-P-X (SEQ ID NO:44), wherein X denotes any amino acid; and,
b) D-C-X₁-X₂-S-G (SEQ ID NO:76), wherein X₁ denotes glutamine, valine, proline, glutamic acid, isoleucine, methionine, or threonine and X₂ denotes any amino acid.

2. The isolated polynucleotide of claim 1, wherein the sequence domain is X₁-C-X₂-E-S-G-L-S-X₃-R-X₄-F-X₅-P-X₆ (SEQ ID NO:45), and wherein X₁ denotes aspartic acid, lysine, glutamic acid, asparagine, serine, glycine, proline or arginine; X₂ denotes asparagine, alanine, serine, glycine, glutamine, valine, proline, glutamic acid, isoleucine, methionine, threonine, or arginine; where X₃ denotes isoleucine, methionine, phenylalanine, glycine, serine, or valine; where X₄ denotes methionine, alanine, serine, glycine, glutamine, leucine, valine, or isoleucine; where X₅ denotes threonine, alanine, valine, isoleucine, proline, leucine, or glycine; and where X₆ denotes isoleucine, leucine, cysteine, alanine, phenylalanine or methionine.

3. The isolated polynucleotide of claim 1, wherein said polynucleotide encodes an EPSP synthase polypeptide that is resistant to glyphosate herbicide.

4. The isolated polynucleotide of claim 1, wherein the sequence domain corresponds to amino acid positions 85 through 99 of SEQ ID NO:2 and is selected from the group consisting of the corresponding positions of SEQ ID NO:5-43 and SEQ ID NO:56-65.

5. The isolated polynucleotide of claim 4, wherein said polynucleotide encodes an EPSP synthase polypeptide having at least 70% sequence identity to the amino acids corresponding to positions 1 through 84 and positions 100 through 431 of SEQ ID NO:2, wherein said EPSP synthase polypeptide is resistant to glyphosate herbicide.

6. The isolated polynucleotide of claim 1 in which the polynucleotide encodes a fusion polypeptide comprising an amino-terminal chloroplast transit peptide and the EPSP synthase enzyme.

7. The isolated polynucleotide of claim 1, wherein said polynucleotide is a synthetic sequence that has been designed for expression in a plant.

8. The isolated polynucleotide of claim 7, wherein said polynucleotide is selected from the group consisting of SEQ ID NO:3, 4, 66, 67, 74, and 75.

9. A method of producing a genetically transformed plant which exhibits glyphosate tolerance, comprising the steps of:
a) inserting into the genome of a plant cell a polynucleotide other than SEQ ID NO:1 and 46 encoding a glyphosate tolerance EPSP synthase polypeptide having a sequence domain selected from the group consisting of:
i) X-C-X-E-S-G-L-S-X-R-X-F-X-P-X (SEQ ID NO:44), wherein X denotes any amino acid; and,
ii) D-C-X₁-X₂-S-G (SEQ ID NO:76), wherein X₁ denotes glutamine, valine, proline, glutamic acid, isoleucine, methionine, or threonine and X₂ denotes any amino acid;
b) obtaining a transformed plant cell; and,
c) regenerating from the transformed plant cell a genetically transformed plant which has increased tolerance to glyphosate herbicide.

10. The method of claim 9, wherein the sequence domain is X₁-C-X₂-E-S-G-L-S-X₃-R-X₄-F-X₅-P-X₆ (SEQ ID NO:45), and wherein X₁ denotes aspartic acid, lysine, glutamic acid, asparagine, serine, glycine, proline or arginine; X₂ denotes asparagine, alanine, serine, glycine, glutamine, valine, proline, glutamic acid, isoleucine, methionine, threonine, or arginine; where X₃ denotes isoleucine, methionine, phenylalanine, glycine, serine, or valine; where X₄ denotes methionine, alanine, serine, glycine, glutamine, leucine, valine, or isoleucine; where X₅ denotes threonine, alanine, valine, isoleucine, proline, leucine, or glycine; and where X₆ denotes isoleucine, leucine, cysteine, alanine, phenylalanine or methionine.

11. The method of claim 9, wherein the sequence domain corresponds to positions 85 through 99 of SEQ ID NO:2 and is selected from the group consisting of the corresponding positions of SEQ ID NO:5-43 and SEQ ID NO:56-65.

12. The method of claim 11, wherein the polynucleotide encodes an EPSP synthase polypeptide having at least 70% sequence identity to the amino acids corresponding to positions 1 through 84 and positions 100 through 431 of SEQ ID NO:2, wherein said EPSP synthase polypeptide is resistant to glyphosate herbicide.

13. The method of claim 9 in which the polynucleotide encodes a fusion polypeptide comprising an amino-terminal chloroplast transit peptide and the EPSP synthase enzyme.

14. The method of claim 9, wherein said polynucleotide is a synthetic sequence that has been designed for expression in a plant.

15. The method of claim 14, wherein said polynucleotide is selected from the group consisting of SEQ ID NO:3, 4, 66, 67, 74, and 75.

16. A glyphosate-tolerant plant cell comprising a heterologous polynucleotide other than SEQ ID NO:1 and 46 encoding a glyphosate tolerance EPSP synthase polypeptide having a sequence domain selected from the group consisting of:
a) X-C-X-E-S-G-L-S-X-R-X-F-X-P-X (SEQ ID NO:44), wherein X denotes any amino acid; and,
b) D-C-X₁-X₂-S-G (SEQ ID NO:76), wherein X₁ denotes glutamine, valine, proline, glutamic acid, isoleucine, methionine, or threonine and X₂ denotes any amino acid.

17. The glyphosate-tolerant plant cell of claim 16, wherein the sequence domain is X₁-C-X₂-E-S-G-L-S-X₃-R-X₄-F-X₅-P-X₆ (SEQ ID NO:45), and wherein X₁ denotes aspartic acid, lysine, glutamic acid, asparagine, serine, glycine, proline or arginine; X₂ denotes asparagine, alanine, serine, glycine, glutamine, valine, proline, glutamic acid, isoleucine, methionine, threonine, or arginine; where X₃ denotes isoleucine, methionine, phenylalanine, glycine, serine, or valine; where X₄ denotes methionine, alanine, serine, glycine, glutamine, leucine, valine, or isoleucine; where X₅ denotes threonine, alanine, valine, isoleucine, proline, leucine, or glycine; and where X₆ denotes isoleucine, leucine, cysteine, alanine, phenylalanine or methionine.

18. The glyphosate-tolerant plant cell of claim 17, wherein the sequence domain corresponds to positions 85 through 99 of SEQ ID NO:2 and is selected from the group consisting of the corresponding positions of SEQ ID NO:5-43 and SEQ ID NO:56-65.

19. The glyphosate-tolerant plant cell of claim 18, wherein the polynucleotide encodes an EPSP synthase polypeptide having at least 70% sequence identity to the amino acids corresponding to positions 1 through 84 and positions 100 through 431 of SEQ ID NO:2, wherein said EPSP synthase polypeptide is resistant to glyphosate herbicide.

20. The glyphosate-tolerant plant cell of claim 16 in which the polynucleotide encodes a fusion polypeptide comprising an amino-terminal chloroplast transit peptide and the EPSP synthase enzyme.

21. The glyphosate-tolerant plant cell of claim 16, wherein said polynucleotide is a synthetic sequence that has been designed for expression in a plant.

22. The glyphosate-tolerant plant cell of claim 21, wherein said polynucleotide is selected from the group consisting of SEQ ID NO:3, 4, 66, 67, 74, and 75.

23. The glyphosate-tolerant plant cell of claim 16 selected from the group consisting of corn, wheat, rice, barley, soybean, cotton, sugarbeet, oilseed rape, canola, flax, sunflower, potato, tobacco, tomato, alfalfa, poplar, pine, eucalyptus, apple, lettuce, peas, lentils, grape and turf grasses.

24. A glyphosate-tolerant plant comprising the plant cell of claim 16.

25. A transformed seed comprising the polynucleotide of claim 1.

26. The glyphosate-tolerant plant of claim 25 selected from the group consisting of corn, wheat, rice, barley, soybean, cotton, sugarbeet, oilseed rape, canola, flax, sunflower, potato, tobacco, tomato, alfalfa, poplar, pine, eucalyptus, apple, lettuce, peas, lentils, grape and turf grasses.

27. A method for selectively controlling weeds in a field containing a plant having planted seeds or plants comprising the steps of:
a) planting the seeds or plants which are glyphosate-tolerant as a result of a polynucleotide other than SEQ ID NO:1 and 46 being inserted into the seed or plant, said polynucleotide encoding a glyphosate tolerance EPSP synthase polypeptide having a sequence domain selected from the group consisting of:
i) X-C-X-E-S-G-L-S-X-R-X-F-X-P-X (SEQ ID NO:44), wherein X denotes any amino acid; and,
ii) D-C-X₁-X₂-S-G (SEQ ID NO:76), wherein X₁ denotes glutamine, valine, proline, glutamic acid, isoleucine, methionine, or threonine and X₂ denotes any amino acid; and,
b) applying to the plants and weeds in a field an effective concentration of glyphosate herbicide to control weeds without significantly affecting the plants.

28. The method of claim 27, wherein said sequence domain is X₁-C-X₂-E-S-G-L-S-X₃-R-X₄-F-X₅-P-X₆ (SEQ ID NO:45), and wherein X₁ denotes aspartic acid, lysine, glutamic acid, asparagine, serine, glycine, proline or arginine; X₂ denotes asparagine, alanine, serine, glycine, glutamine, valine, proline, glutamic acid, isoleucine, methionine, threonine, or arginine; where X₃ denotes isoleucine, methionine, phenylalanine, glycine, serine, or valine; where X₄ denotes methionine, alanine, serine, glycine, glutamine, leucine, valine, or isoleucine; where X₅ denotes threonine, alanine, valine, isoleucine, proline, leucine, or glycine; and where X₆ denotes isoleucine, leucine, cysteine, alanine, phenylalanine or methionine.

29. The method of claim 28, wherein the sequence domain corresponds to positions 85 through 99 of SEQ ID NO:2 and is selected from the group consisting of the corresponding positions of SEQ ID NO:5-43 and SEQ ID NO:56-65.

30. The method of claim 29, wherein the polynucleotide encodes an EPSP synthase polypeptide having at least 70% sequence identity to the amino acids corresponding to positions 1 through 84 and positions 100 through 431 of SEQ ID NO:2, wherein said EPSP synthase polypeptide is resistant to glyphosate herbicide.

31. The method of claim 28 in which the polynucleotide encodes a fusion polypeptide comprising an amino-terminal chloroplast transit peptide and the EPSP synthase enzyme.

32. The method of claim 28, wherein said polynucleotide is a synthetic sequence that has been designed for expression in a plant.

33. The method of claim 32, wherein said polynucleotide is selected from the group consisting of SEQ ID NO:3, 4, 66, 67, 74, and 75.

34. An isolated nucleic acid molecule that encodes a glyphosate tolerance EPSPS enzyme, wherein said glyphosate tolerance EPSPS enzyme has a Kᵢ (glyphosate)/Kₘ (PEP) between about 100 and about 1700.

35. A plant having stably incorporated into its genome a DNA construct comprising a nucleotide sequence that encodes a glyphosate tolerance EPSPS polypeptide, said EPSPS polypeptide having a Kᵢ (glyphosate)/Kₘ (PEP) between about 100 and about 1700, said plant exhibiting tolerance to glyphosate herbicide.

36. The plant of claim 35, wherein said plant is selected from the group consisting of corn, wheat, rice, barley, soybean, cotton, sugarbeet, oilseed rape, canola, flax, sunflower, potato, tobacco, tomato, alfalfa, poplar, pine, eucalyptus, apple, lettuce, peas, lentils, grape and turf grasses.
